(19) United States
(12) Patent Application Publication
(10) Pub. No.: US 2022/0160779 A1
(43) Pub. Date: May 26, 2022

(54) COMPOSITION FOR PROMOTING CARTILAGE TISSUE REGENERATION

(71) Applicant: National University Corporation Tokyo Medical and Dental University, Tokyo (JP)

(72) Inventors: Kunikazu TSUJI, Bunkyo-ku, Tokyo (JP); Miyoko OJIMA, Bunkyo-ku, Tokyo (JP); Takeshi MUNETA, Bunkyo-ku, Tokyo (JP); Hideyuki KOGA, Bunkyo-ku, Tokyo (JP); Ichiro SEKIYA, Bunkyo-ku, Tokyo (JP)

(73) Assignee: National University Corporation Tokyo Medical and Dental University, Tokyo (JP)

(21) Appl. No.: 17/598,049

(22) PCT Filed: Mar. 19, 2020
(86) PCT No.: PCT/JP2020/012229

§ 371 Date: Sep. 24, 2021

(30) Foreign Application Priority Data
Mar. 26, 2019 (JP) 2019-059418

(51) Int. Cl. A61K 35/28; A61K 38/17; A61L 27/22; A61L 27/38; A61P 19/02; A61P 19/08

(52) U.S. Cl. CPC A61K 35/28; A61K 38/17; A61P 19/08; A61L 27/38; A61P 19/02; A61L 27/22

(57) ABSTRACT

The present inventors have clarified that activin has an activity of promoting the proliferation of MSCs and an activity of promoting the regeneration of meniscus or cartilaginous tissue without affecting differentiation multipotency. Further, the present inventors have also found that the use of activin makes it possible to treat and prevent meniscus damage or cartilage disorders, including suppression of degenerative degeneration of cartilage and pain.

### TECHNICAL FIELD

The present invention relates to a composition for promoting regeneration of cartilaginous tissues, a composition for promoting proliferation of mesenchymal stem cells, and a composition for treating or preventing meniscus damage or cartilage disorders, containing activin as an active ingredient. The present invention also relates to a method for treating or preventing meniscus damage or cartilage disorders using activin, and a method for predicting prognosis of a cartilage disorder in a joint using the joint activin level as an indicator.

### BACKGROUND ART

Cartilaginous tissue is a connective tissue composed of extracellular matrix and chondrocytes scattered therein, and its functions include shock mitigation and almost frictionless joint movement. Also, the superficial layer of this tissue is covered by perichondrium, which prevents vascularization, and therefore, compared to other tissues with blood vessels, it is difficult to receive physiologically active substances and the like that are useful for regeneration. Thus, this tissue is known to have an extremely poor regenerative capacity. Therefore, when the cartilage of joints is damaged due to aging, obesity, strenuous exercise, injury, disease, and the like, the damaged parts are not fully repaired and develop into diseases such as osteoarthritis (OA), which is accompanied by joint pain and loss of joint functions (such as cartilage degeneration, meniscus damage, and ligament disorder).

Treatment strategies for OA and other cartilage disorders are based on symptom management with anti-inflammatory and analgesic drugs, programmed exercise to improve joint activity and stability, weight control, and the like. However, such treatments cannot be expected to restore articular cartilage, and most patients cannot be spared from worsening of the symptoms.

This has led to intense research in the field of cartilage metabolism to develop new therapeutic methods, and its progress has led to the discovery of novel drug targets (DMOADs: Disease Modifying OA Drugs) that play a role in improving both symptoms and degenerative changes in arthritic joints.

For example, by intra-articular administration, FGF18 has been shown to suppress articular cartilage degeneration and increase cartilage thickness at the tibial plateau in a rat meniscus damage model (NPL 1). Moreover, recombinant FGF18 protein (rhFGF18, trade name: Sprifermin) has already been tested in first-in-human studies in the United States, and intra-articular injection of Sprifermin has been shown to have the effects of suppressing degenerative degeneration of articular cartilage, increasing cartilage thickness, and reducing joint pain (NPLs 2 to 4). However, FGF18 has originally been shown to function in a pro-osteogenic manner (NPLs 5 to 7), and there is concern about the increased risk of endochondral ossification, such as the formation of osteophytes, when a recombinant protein is injected into the joint.

From another viewpoint, with regard to a novel therapeutic method for articular cartilage disorders, regenerative medicine for OA patients has recently been developed by the present inventors, in which autologous mesenchymal stem cells (MSCs) are cultured to expand in vitro, and then transplanted back into the patient's knee joint. Specifically, the present inventors report that the synovium contains multipotent MSCs and has a higher differentiation potential into chondrocytes compared to those present in other tissues such as bone marrow, skeletal muscle, and infrapatellar fat pad (IFP) (NPLs 8 to 10).

On the other hand, in-vitro studies have reported a relationship between activin and the proliferation of undifferentiated mesenchymal cells and the promotion of the process of endochondral ossification in vivo, since siRNA against Activin A suppresses chondrogenic differentiation (NPL 11), promoted chondrogenic differentiation is observed with the administration of activin-BMP2 chimeric ligand (NPL 12), activin-BMP2 chimeric ligand (AB235) promotes the formation of cartilage-like tissues (NPL 13), activin administration promotes the expression of proteoglycans (NPL 14), activin promotes the proliferation of rheumatoid arthritis-derived synovial cells (mesenchymal cells) (NPL 15), and the like.

### CITATION LIST

### Non Patent Literature

[NPL 1] Moore E E. et al., Osteoarthritis Cartilage, 2005, vol. 13, Issue 7, pp. 623 to 631

[NPL 2] Dahlberg L E. et al., Clin Exp Rheumatol, 2016, vol. 34, Issue 3, pp. 445 to 450

[NPL 3] Lohmander L S. et al., Arthritis Rheumatol (Hoboken, N.J.), 2014, vol. 66, Issue 7, pp. 1820 to 1831

[NPL 4] Eckstein F. et al., Arthritis Rheumatol (Hoboken, N.J.), 2015, vol. 67, Issue 11, pp. 2916 to 2922

[NPL 5] Hamidouche Z, et al., J Cell Physiol, 2010, vol. 224, Issue 2, pp. 509 to 515

[NPL 6] Jeon E. et al., PLoS One, 2012, 7(8): e43982

[NPL 7] Nagayama T. et al., Congenit Anom, 2013, vol. 53, Issue 2, pp. 83 to 88

[NPL 8] Sekiya I. et al., Clin Orthop Relat Res, 2015, vol. 473, Issue 7, pp. 2316 to 2326

[NPL 9] Koga H. et al., Cell Tissue Res., 2008, vol. 333, Issue 2, pp. 207 to 215

[NPL 10] Segawa Y. et al., J Orthop Res., 2009, vol. 27, Issue 4, pp. 435 to 441

[NPL 11] Djouad F. et al., Stem Cell Research & Therapy, 2010, 1:11

[NPL 12] Peran M. et al., Stem Cell Research, 2013, vol. 10, pp. 464 to 476

[NPL 13] Jimenez G. et al., Scientific Reports, 2015, 5: 16400

[NPL 14] Luyten F. et al., Experimental Cell Research, 1994, vol. 210, Issue 2, pp. 224 to 229

[NPL 15] Ota F. et al., Arthritis & Rheumatism, 2003, vol. 48, Issue 9, pp. 2442 to 2449

### SUMMARY OF INVENTION

### Technical Problem

An object of the present invention is to provide a substance and a method that promote the regeneration of cartilage tissues and, by extension, enable the treatment of cartilage disorders and the like.

### Solution to Problem

As described above, the present inventors have been working diligently to develop a therapeutic method for cartilage disorders such as OA by autologous transplantation of synovium-derived MSCs. Additionally, the application of synovial MSCs to autologous MSC transplantation therapy to regenerate articular cartilage and/or meniscus began in 2012 at the university hospital to which the present inventors belong, and considerable results have been obtained in the first set of clinical trials.

Meanwhile, the present inventors found that there were individual differences among patients in the cell proliferative capacity for in-vitro culture of MSCs for transfer and in the regenerative effect of cartilage after MSC transplantation. Then, the present inventors hypothesized that these differences were due to differences in the amount of substance involved in the proliferation and/or viability of MSCs in the synovial joint. The present inventors also thought that if such substance was successfully identified, it would be possible to simply and easily activate MSCs in synovial joints, and cartilage regeneration, thus the treatment of cartilage disorders, would become more effective. Based on the aforementioned hypothesis, the present inventors tried to identify and characterize the bioactive substances involved in the proliferative potential of MSCs in synovial joints.

Specifically, synovial fluids obtained from patients who had undergone anterior cruciate ligament reconstruction (ACL-R) were divided into two groups based on the proliferative activity of MSCs, and the expression levels of 174 proteins in each synovial fluid group were analyzed and compared. As a result, the present inventors succeeded in identifying Activin A as a protein with high expression levels in the synovial fluid showing high pro-proliferative activity of MSCs.

Then, the present inventors further investigated the relationship between the protein and MSC proliferation, and found that activin could promote MSC proliferation without affecting the expression of MSC-related cell surface antigens and differentiation multipotency in vitro.

In addition, the results of in-vivo (animal) experiments showed that injection of Activin A into the joint significantly promoted the regeneration process after the resection of the anterior half of medial meniscus. Furthermore, the present inventors also found that Activin A significantly suppressed the degenerative degeneration of cartilaginous tissues around the defect site in a model of total articular cartilage loss. Furthermore, the present inventors also found that in a mono-iodoacetic acid (MIA)-induced arthritis model, pain associated with arthritis was also significantly relieved by Activin A, indicating that activin was extremely effective in improving and treating the symptoms of cartilage disorders through cartilage regeneration.

Moreover, in a retrospective human study, the present inventors also found that the protein level of Activin A in synovial fluid after ACL-R surgery was positively correlated with the degree of its recovery, that is, the expression level of activin A in the joint could be a good indicator of cartilaginous tissue regeneration after joint injury. Thus, the present invention has been completed.

The present invention relates to a composition for promoting regeneration of cartilaginous tissues, a composition for promoting proliferation of mesenchymal stem cells, and a composition for treating or preventing cartilage disorders, containing activin as an active ingredient. The present invention also relates to a pharmaceutical composition for treating and/or preventing meniscus damage or cartilage disorder, containing activin as an active ingredient. The present invention also relates to a method for treating or preventing cartilage disorders using activin, and a method for predicting prognosis of a cartilage disorder in a joint using the joint activin level as an indicator, and more specifically as follows.

<1> A composition for promoting regeneration of cartilaginous tissues, comprising: activin as an active ingredient.
<2> A composition for promoting proliferation of mesenchymal stem cells, comprising: activin as an active ingredient.
<3> The composition according to <1> or <2>, which is a composition for treating or preventing a cartilage disorder.
<4> The composition according to <3>, wherein the cartilage disorder is due to damage or defect of a meniscus.
<5> The composition according to <3>, wherein the cartilage disorder is due to defect or degenerative degeneration of articular cartilage.
<6> A pharmaceutical composition for treating and/or preventing meniscus damage or cartilage disorder, comprising: activin as an active ingredient.
<7> The composition according to <6>, wherein the meniscus damage is meniscus tear, meniscus degeneration, sparing meniscus degeneration, meniscus defect, meniscus damage associated with accident or sports injury, or meniscus damage associated with aging.
<8> The composition according to <6> or <7>, wherein the cartilage disorder is cartilage defect, cartilage damage, cartilage degeneration, cartilage wear, cartilage loss, cartilage degradation, cartilage deformation, defect of articular cartilage, degenerative degeneration of articular cartilage, osteoarthritis, degenerative knee arthropathy, degenerative hip arthropathy, deformative shoulder arthropathy, traumatic cartilage damage, traumatic cartilage defect, ligamentous damage complication, osteochondritis dissecans, aseptic necrosis, or intervertebral disc damage.
<9> The pharmaceutical composition according to any one of <6> to <8> for administration before, during, and/or after meniscal suture or meniscal repair.
<10> The pharmaceutical composition according to any one of <6> to <9>, wherein the activin is Activin A.
<11> The pharmaceutical composition according to any one of <6> to <10>, which is used in combination with mesenchymal stem cells.
<12> A method for treating or preventing cartilage disorders, comprising the step of: administering activin to a subject's cartilage disorder site.
<13> The method according to <12>, further comprising the step of: administering mesenchymal stem cells to the cartilage disorder site.
<14> A method for predicting prognosis of a cartilage disorder in a joint, comprising the steps of:

measuring a concentration of activin in a joint of a subject; and

predicting that the prognosis of the cartilage disorder in the subject is good if the concentration obtained in the step is higher than a reference value.

The pharmaceutical composition of the present invention includes activin-containing therapeutic and/or prophylactic agents for meniscus damage or cartilage disorders. The present invention also relates to the use of activin for the production of pharmaceutical compositions for the prevention and/or treatment of meniscus damage or cartilage disorders, the use of activin for the prevention and/or treatment of meniscus damage or cartilage disorders, activin for use in the prevention and/or treatment of meniscus damage or cartilage disorders, and a method for preventing and/or treating meniscus damage or cartilage disorders, including administering an effective amount of activin to a subject. Note that the “subject” is a human or other animal in need of that prevention or treatment, and in one embodiment, a human in need of that prevention or treatment.

### Advantageous Effects of Invention

According to the present invention, it is possible to promote the proliferation of MSCs and promote the regeneration of the meniscus or cartilage, as well as treat or prevent meniscus damage or cartilage disorders. Furthermore, it is possible to predict the prognosis of cartilage disorders.

### BRIEF DESCRIPTION OF DRAWINGS

FIG. 1A is a diagram schematically showing a method for preparing synovial fluid collected from a patient immediately before and 3 to 4 days after anterior cruciate ligament reconstruction (ACL-R), and the experiments whose results are shown in FIGS. 2A and 2B.

FIG. 1B is a diagram schematically showing a method for preparing synovial fluid collected from a patient 3 to 4 days after ACL-R, used in the experiments whose results are shown in FIGS. 2C, 2D, 3A, 3B, 3C, 4A, 4B, 5A, and 5B.

FIG. 2A is a dot-plot diagram showing the results of measuring the cell count after adding synovial fluid at a ratio of 14% to the MSC culture system and culturing for 2 weeks. In the figure, “Pre” shows the result of adding synovial fluid collected from patients immediately before ACL-R, and “At 3-4 days after ACL-R” shows the results of adding synovial fluid collected from the patients 3 to 4 days after ACL-R.

FIG. 2B is a dot-plot diagram showing the results of measuring the cell count after adding each of the synovial fluids before and after heat-inactivation treatment at a ratio of 14% to the MSC culture system and culturing for 2 weeks.

FIG. 2C is a dot-plot diagram showing the results of analyzing the interrelationship between the concentration of each complement protein (C5a, C3a, and C4a) in the synovial fluid and the MSC cell count after 2 weeks of culture using normal medium with the synovial fluid added at a ratio of 14%.

FIG. 2D is a dot-plot diagram showing the relationship between the concentration of each of PDGF (Platelet Derived Growth Factor)-AA and PDGF-BB in the synovial fluid and the number of MSCs after culture in the presence of the synovial fluid.

FIG. 3A is a dot-plot diagram showing the results of quantifying cytokines and chemokines in the synovial fluid before (pre) and 3 to 4 days after ACL-R surgery (3-4 days after ACL-R) by ELISA (Enzyme-linked Immunosorbent Assay). In the figure, the vertical axis indicates the concentration of cytokines and chemokines (pg/ml).

FIG. 3B is a dot-plot diagram showing the relationship between the concentration of cytokines and chemokines each in the synovial fluid and the number of MSCs after culture in the presence of the synovial fluid. In the figure, the vertical axis shows the concentration of cytokines and chemokines each, and the horizontal axis shows the number of MSCs after culture in the presence of the synovial fluid.

FIG. 3C is a graph showing the results of culturing MSCs in normal growth medium with the addition of IL6, IL8, and IL10 recombinant proteins in amounts shown in the figure, and analyzing their proliferation using MTT assay. The control group used was normal growth medium (without cytokines, “10% FBS” in the figure). The comparison group used was media obtained by adding cytokines and chemokines each 10 and 100 times the ED50 (the concentration of cytokines at which the activity is 50% of the maximum response) to the normal growth medium (in each graph, “10% FBS+ . . . ED50×10” and “10% FBS+ . . . ED50×100”). The vertical axis of the figure shows the absorbance at 560 nm, which is directly proportional to the cell count, detected in the MTT assay, and the absorbance increases as the cell count increases.

FIG. 4A is a heat map showing the results of dividing the synovial fluid collected in the ACL-R into two groups based on the proliferative activity of MSCs (Low, high), comparing the protein concentrations in each synovial fluid group using an antibody array that can simultaneously evaluate the expression of 174 different proteins, and selecting 13 proteins with significant differences (by u-test) in expression levels between the two groups.

FIG. 4B is a graph showing the concentrations of the 13 proteins in the two groups. In the figure, the vertical axis shows the concentration of each protein in the synovial fluid (pg/ml, mean value and standard deviation (SD)).

FIG. 5A is a dot-plot diagram showing the relationship between the concentration of Activin A in the synovial fluid and the synovial fluid derived from the patients. In the figure, “Pre” indicates synovial fluid collected from patients immediately before ACL-R, and “At 3-4 days after ACL-R” indicates synovial fluid collected from the patients 3 to 4 days after ACL-R. The vertical axis shows the concentration of Activin A.

FIG. 5B is a dot-plot diagram showing the relationship between the concentration of Activin A in the synovial fluid and the number of MSCs after 2 weeks of culture using normal growth medium with the synovial fluid added at a ratio of 14%. In the figure, the vertical axis shows the concentration of Activin A, and the horizontal axis shows the number of MSCs after culture in the presence of the synovial fluid.

FIG. 5C is a graph showing the change over time in the number of MSCs cultured in normal growth medium without Activin A (“10% FBS” in the figure) and normal growth medium containing Activin A recombinant protein (rh Activin A) (in each graph, “10% FBS+rh Activin A ED50×10” and “10% FBS+rh Activin A ED50×100”). Note that the notation in the figure is the same as that in FIG. 3C.

FIG. 5D is a graph showing the change over time in the number of MSCs cultured in serum low-concentration proliferation medium without Activin A (“0.5% FBS” in the figure) and rh Activin A-containing serum low-concentration proliferation medium (in each graph, “0.5% FBS+rh Activin A ED50×10” and “0.5% FBS+rh Activin A ED50×100”). Note that the notation in the figure is the same as that in FIG. 3C.

FIG. 5E is a graph showing the change over time in the number of MSCs cultured in serum low-concentration proliferation medium without Activin A (“0.5% FBS” in the figure), rh Activin A-containing serum low-concentration proliferation medium (in each graph, “0.5% FBS+rh Activin A”), and each inhibitor (Akt, Ku0063794, LY294002, or PD98050) and rh Activin A-containing serum low-concentration proliferation medium (in each graph, “0.5% FBS+rh Activin A+each inhibitor”). Note that the notation in the figure is the same as that in FIG. 3C.

FIG. 5F is a photograph showing the results of Western blotting to detect the phosphorylation of each protein in MSCs after the addition of rh Activin A and the like to the medium. In the figure, “minute (min)” indicates the time elapsed after the addition of rh Activin A and the like to the medium. “A,” “P,” “AP,” and “N” indicate the results after the addition of rh Activin A, the results after the addition of PDGF-AA, the results after the addition of rh Activin A and PDGF-AA, and the results without these bioactive substances, respectively. The “pAkt” and “ppAkt” indicate the results of detecting the non-phosphorylated and phosphorylated states of Akt protein, respectively. The “p44/42” and “pp44/42” indicate the results of detecting the non-phosphorylated and phosphorylated states of p44/42 MAPK (Erk1/2) protein, respectively.

FIG. 6A is a graph showing the results of analyzing MSCs that are positive for the stem cell antigens CD73, CD90, CD105, and CD44 using a flow cytometer. In the figure, “rh Activin A” indicates the results of analyzing MSCs cultured in the presence of rh Activin A for 2 weeks, and “Control” indicates the results of analyzing MSCs cultured in the absence of rh Activin A. The vertical axis shows the positive rate of each antigen. In one experiment, 10000 cells were scanned, and among them, the abundance ratio of cells expressing the target antigen was shown as a percentage. Four independent experiments were performed, and the figure shows the mean value and SD.

FIG. 6B is a micrograph showing the results of inducing differentiation of MSCs into cartilage, bone, and adipocytes in vitro after 2 weeks of culture in the presence (“rh Activin A” in the figure) or absence (“control” in the figure) of rh Activin A. In the figure, “Macro” indicates the result of observing the induced formation of chondrocyte aggregates. The “Toluidine Blue” indicates the result of detecting the cartilage matrix by staining sections of the aggregates with toluidine blue. “Calcification” indicates the result of detecting colonies producing calcified bone matrix by staining calcium deposits in the extracellular matrix with alizarin red. “Adipogenesis” indicates the result of inducing adipogenesis, staining the colonies with Oil Red O solution, and detecting adipose tissues.

FIG. 6C is a diagram showing the results of inducing differentiation of MSCs into chondrocyte aggregates in vitro, as in FIG. 6B. In the figure, “rh Activin A,” “rh PDGF-BB,” and “rh Activin A/rh PDGF-BB” indicate the results of measuring the wet weights of the chondrocyte aggregates obtained as a result of pellet culture for 2 weeks using chondrogenic differentiation induction medium supplemented with rh Activin A, rh PDGF-BB (recombinant PDGF-BB protein), or rh Activin A and rh PDGF-BB, respectively. The control shows the results of forming chondrogenic aggregates by using chondrogenic differentiation induction medium normally. The figure shows the mean value and SD, and the number of spheroids subjected to the experiment (n number) is shown in the parentheses on the horizontal axis. The picture at the center shows the results of observing the appearance of each chondrogenic spheroid. In the histological images, each of the upper photographs shows the results of observation by staining sections of the spheroid with hematoxylin/eosin, and each of the lower photographs shows the results of observation by staining sections of the spheroid with toluidine blue.

FIG. 7A is a photograph showing the results of administering rh Activin A and the like (dose: 42 ng/knee) at day 7 after resection of the anterior ½ of the mouse medial meniscus, and observing the articular tissues 3 days later (day 10 after meniscectomy). In the figure, “rh Activin A” indicates a model with rh Activin A administered thereto. For comparison, “rh PDGF-AA” indicates a model with rh PDGF-AA (recombinant PDGF-AA protein) administered thereto, “rh PDGF-AA+rh Activin A/rh PDGF-AA” indicates a model with rh Activin A and rh PDGF-AA simultaneously administered thereto, and “PBS/Control” indicates a model without bioactive substance administered thereto, but with only a vehicle (phosphate buffered saline) administered thereto. The schematic diagrams at the bottom right illustrate the photographs in the histological images, and in each group, “Macro” shows a weakly expanded photograph of the result of observing a section of the joint stained with Safranin O/Fast Green. The upper part of the picture is the distal end of the femur and the lower part is the proximal end of the tibia, and the left side is anterior and the right side is posterior. The strong magnification of the region surrounded by the square in the figure (meniscus regeneration part) is the other four panels. “SafO” shows the accumulation site (Safranin O staining) of cartilage matrix (proteoglycan) in the regenerated meniscus region (surrounded by a square), and “HE” shows the distribution of cells in the meniscus regeneration region (hematoxylin/eosin staining). “Col I IHC” and “Col II IHC” indicate the results of immunohistochemical staining (IHC) of sections of the meniscus regeneration region (the area surrounded by a square) for type I and type II collagen, respectively.

FIG. 7B is a photograph showing the results of administering rh Activin A and the like at day 7 after meniscectomy in the medial meniscus anterior ½ resection model, and observing the articular tissues 14 days later (day 21 after meniscectomy). The notation in the figure is the same as that in FIG. 7A.

FIG. 7C is a photograph showing the results of administering rh Activin A and the like at day 7 after meniscectomy in the medial meniscus anterior ½ resection model, and observing the articular tissues 49 days later (day 56 after meniscectomy). The notation in the figure is the same as that in FIG. 7A.

FIG. 7D is a photograph showing the results of administering rh Activin A and the like at day 7 after meniscectomy in the medial meniscus anterior ½ resection model, and observing the articular tissues 77 days later (day 84 after meniscectomy). The notation in the figure is the same as that in FIG. 7A.

FIG. 7E is a graph showing the results of administering rh Activin A and the like at day 7 after meniscectomy, and at day 10 (10 d), day 21 (3 W), day 56 (8 W), and day 84 (12 W) after meniscectomy, evaluating the degree of meniscus regeneration using the modified Pauli's score, in the medial meniscus anterior ½ resection model. The notation in the figure is the same as that in FIG. 7A.

FIG. 8 is a photograph showing the results of intra-articular injection of rh Activin A (dosage: 180 ng/knee) or rrPDGF-AA (dosage: 900 ng/knee) 1 week after total articular cartilage loss in a rat model of total articular cartilage loss, and observation of the articular tissues 3 weeks later (week 4 after total articular cartilage loss). In the figure, “Saf O” and “Tol Blue” indicate the results of observing sections of the articular tissues stained with Safranin O and Toluidine Blue, respectively.

FIG. 9A is a schematic diagram showing an experimental scheme to study the pain-relieving effect of rh Activin A administration in a mono-iodoacetic acid (MIA)-induced arthritis model.

FIG. 9B is a graph showing the results of measuring the laterality in the load of the rat hindlimbs in the MIA-induced arthritis model. In the figure, “rh Activin A” indicates the results of analyzing a rat in which MIA was injected into the left knee joint, and 14 days later, rh Activin A was injected into the same joint (dose: 200 ng/knee). The “control” shows the results of analyzing a rat in which MIA was injected into the left knee joint, and 14 days later, instead of rh Activin A, its vehicle, PBS, was injected into the same joint.

FIG. 10 is a dot-plot diagram showing the relationship between the concentration of Activin A in the synovial fluid of patients at days 3 to 4 after ACL-R and the patients' standing knee recovery score (IKDC score) 2 months after surgery for those patients. Note that for the evaluation of IKDC scores, refer to the knee evaluation form of the International Knee Documentation Committee (IKDC) (https://www.sportsmed.org/AOSSMIMIS/members/downloads/research/IKDCJapanese.pdf>.

### DESCRIPTION OF EMBODIMENTS

(Composition Containing Activin as Active Ingredient)

As shown in Examples described later, the present inventors have clarified that Activin A promotes the proliferation of MSCs without affecting the differentiation multipotency in vitro. Furthermore, as a result of verification using a mouse model of regeneration after meniscectomy and a rat model of total articular cartilage loss, it has also been clarified that administration of Activin A induces regeneration of meniscus tissues, promotes regeneration of cartilaginous tissues in the damaged area, and suppresses degenerative degeneration of normal cartilage around the damaged cartilage. Furthermore, in an arthritis-induced knee pain model using rats, it has also been clarified that intra-articular administration of Activin A results in mitigation of pain avoidance behavior of rats.

Therefore, the present invention provides a composition for promoting the proliferation of MSCs, a composition for promoting regeneration of cartilaginous tissues, a composition for suppressing the degenerative degeneration of cartilaginous tissues, and a composition for treating or preventing meniscus damage or cartilage disorders, containing activin as an active ingredient.

“Activin” means a homodimer (Activin A, Activin B) or a heterodimer (Activin AB) of two inhibin β chains (βA, βB chains). The inhibin βA chain is, for example, a polypeptide composed of the amino acid sequence specified by NCBI accession number: NP_002183, and the inhibin βB chain is, for example, a polypeptide composed of the amino acid sequence specified by NCBI accession number: NP_002184.

In the present invention, “activin” is preferably a homodimer (active Activin A) of the mature inhibin βA chain in which the N-terminal peptide is cleaved, a homodimer (active Activin B) of the mature inhibin βB chain in which the N-terminal peptide is cleaved, or a heterodimer (active Activin AB) of the mature inhibin βA chain in which the N-terminal peptide is cleaved and the mature inhibin βB chain in which the N-terminal peptide is cleaved, more preferably a homodimer of the inhibin βA chain composed of positions 311 to 426 in which the amino acids at positions 1 to 310 on the N-terminal side are removed, a homodimer of the inhibin βb chain composed of positions 293 to 407 in which the amino acids at positions 1 to 292 on the N-terminal side are removed, or a heterodimer of the inhibin βA chain composed of positions 311 to 426 and the inhibin βB chain composed of positions 293 to 407, and further preferably a homodimer in which a fragment of the inhibin βA chain composed of positions 311 to 426 is disulfide-bonded, a homodimer in which a fragment of the inhibin βB chain composed of positions 293 to 407 is disulfide-bonded, or a heterodimer in which a fragment of the inhibin βA chain composed of positions 311 to 426 and a fragment of the inhibin βB chain composed of positions 293 to 407 are disulfide-bonded. Further, the activin of the present invention may be glycosylated or non-glycosylated.

Note that the inhibin β chain according to the present invention is not limited to each polypeptide composed of the amino acid sequence identified by the NCBI accession number, but may also include variants and homologues to the above typical amino acid sequences, as long as the activity is maintained. In addition, such variants and homologues usually have high homology to a typical amino acid sequence. The high homology is usually 60% or more, preferably 70% or more, more preferably 80% or more, further preferably 85% or more, and more preferably 90% or more (for example, 95% or more, 96% or more, 97% or more, 98% or more, and 99% or more).

Activin is known to be produced in many animals and many cell types. Therefore, it can be obtained by separating, extracting, and purifying from these cells and the like according to a known method. Activin can also be biosynthesized by genetic methods using E. coli, animal cells, insect cells, plant cells, cell-free protein synthesis systems (such as reticulocyte extract and wheat germ extract), and the like, based on the information on the DNA sequence encoding the amino acid sequence described above. Furthermore, based on the information on the amino acid sequence, it can be chemically synthesized using a peptide synthesizer or the like. Further, as shown in Examples described later, recombinant proteins of activin are already commercially available. Therefore, such commercially available products (such as Activin Recombinant Protein manufactured by R&D Systems Inc. and Activin A Solution, Human, Recombinant manufactured by Ajinomoto Co., Inc.) are also suitable for use in the present invention.

Furthermore, in the present invention, a substance having activin-like activity can be used instead of activin. Examples of such substances include activin mimetics designed and synthesized based on the structure of activin.

In addition, as shown in Examples described later, activin activates the signal transduction pathway downstream thereof to promote the proliferation of MSCs. Therefore, substances that activate the activin signal transduction pathway (such as proteins, nucleic acids, and low-molecular-weight compounds) and substances that function suppressively on substances for inactivating activin signals (such as proteins, nucleic acids, and low-molecular-weight compounds (inhibitors)) can also be used as substances with the above activin-like activity. The “activin signal transduction pathway” is not particularly limited as long as it is a signal transduction pathway in which activin is involved, and examples thereof include a Pl3K-Akt/PKB signal transduction pathway. In addition, examples of the “substances that activate the activin signal transduction pathway” include intracellular transduction substances activated by activin (intracellular transduction substances that constitute canonical and non-canonical signal transduction pathways, more specifically, Smad2 or complexes of Smad3 and Smad4, and kinases involved in the intracellular MAPK cascade such as TAK1) and activators for those individual proteins. Further, there are inhibitors against Smads (such as Smad6 and Smad7) that acts suppressively on activin signals, and nucleic acids and proteins that function suppressively on them. Examples of such nucleic acids and proteins include siRNA targeting the Smads above, shRNA, RNA having ribozyme activity (ribozyme), dsRNA, genome editing systems, antibodies, and peptides having dominant negative trait.

In addition, activin is known to bind to heterodimers of type I receptors (such as Alk2 and Alk4) and type II receptors (ActRIIa or ActRIIb) as cell membrane receptors and activate intracellular signal transduction pathways. Therefore, substances capable of binding to the receptor heterodimers (such as proteins, nucleic acids, and low-molecular-weight compounds) can also be used as the substance having activin-like activity.

In addition, substances having activin-like activity can also be obtained by selecting from a library of synthetic low-molecular-weight compounds by a screening method using the activity that activin can exhibit as an index.

In the present invention, “activin activity” means the activity and the like of at least one of MSC pro-proliferative activity, cartilage pro-regeneration activity, degenerative degeneration suppressing activity, and pain-relieving effect. Whether the variants or homologues of activin and the low-molecular-weight compounds can exhibit the activity can be evaluated by using an MSC culture system and/or a cartilage damage model animal as shown in Examples described later. Further, in the present invention, the “activin activity” may include not only the new activity of activin found by the present inventors but also known activities that have been known conventionally. Examples of such known activity include hemoglobin synthesis inducibility, and can be evaluated by using, for example, a culture system of human chronic myelogenous leukemia cells (K562 cells) (see Ralph H. Schwall, Cora Lai, Methods in Enzymology, 1991, vol. 198, pp. 340 to 346).

The “mesenchymal stem cell (MSC)” means a somatic stem cell derived from a mesodermal tissue (mesenchyme) and capable of differentiating into a cell belonging to the mesenchymal system. Such cells can be obtained by isolation from bone marrow (Prockop, D. J., 1997, Science. 276: 71-4), mesenchymal tissues, such as synovium (Sekiya I. et al., 2015, Clin Orthop Relat Res, 473(7): 2316 to 2326, De Bari, C. et al., 2001, Arthritis Rheum. 44: 1928-42), periosteum (Fukumoto, T. et al., 2003, Osteoarthritis Cartilage. 11: 55-64), adipose tissue (Zuk, P. A. et al., 2002, Mol Biol Cell. 13: 4279-95), and muscle tissue (Cao et al., 2003, Nat Cell Biol. 5: 640-6). In addition, the “mesenchymal stem cell (MSC)” is derived from humans or non-human animals (such as pigs, cattle, horses, sheep, goats, chickens, ducks, ostriches, domestic ducks, dogs, cats, rabbits, hamsters, mice, rats, and monkeys), and further, may be cells that reside within the body of these animals (such as synovium, intra-articular, synovial fluid, periosteum, adipose tissue, muscle tissue, and bone marrow) or cells that reside outside the body (such as cultured MSCs).

The “cartilaginous tissue” refers to the connective tissue containing chondrocytes or chondrocyte-like cells and extracellular matrix (for example, proteoglycans such as chondroitin sulfate proteoglycans, keratan sulfate proteoglycans, and dermatan sulfate proteoglycans, collagen types I, II, IX, and XI), including vitreous cartilage tissue (articular cartilage tissue), fibrocartilage tissue (such as meniscus), and elastic cartilage tissue. In addition, the “cartilaginous tissue” is derived from human or non-human animals, and further, may be tissue that resides inside the body of these animals (such as joints, meniscus, tendon and ligament attachments, intervertebral discs, thoracic wall, larynx, airway, and ears), or tissue that resides outside the body (such as cultured cartilaginous tissue). Note that in the present invention, the “cartilaginous tissue” is a concept including cells constituting the tissue (such as chondrocytes and chondrocyte-like cells). Also, the “regeneration of cartilage tissue” means the physical and/or functional return of the later-described damaged cartilage tissue to its original normal state or a state close thereto, including cartilaginous tissue repair. In addition, the “degenerative degeneration of cartilaginous tissue” means morphological and quantitative changes in cartilaginous tissue (structural changes such as cartilage abrasion and tears), and qualitative changes in the constituent components of the extracellular matrix (such as collagen and proteoglycans) (for example, changes in the composition ratio of cartilage matrix, such as changes in glycosylation to core proteins, accumulation of AGE (Advanced glycosylation End products), and decrease in proteoglycans) are also included in the degenerative degeneration of cartilaginous tissue. In the present invention, “suppression” includes not only partial suppression but also complete suppression (inhibition).

The “cartilage disorders” refer to conditions in which cartilage tissue is damaged due to aging, trauma, or various other factors, such as conditions of cartilage defect, cartilage damage, cartilage degeneration, cartilage wear, cartilage loss, cartilage degradation, and cartilage deformation. In addition, specific examples of cartilage disorders (diseases) include meniscus damage (including partial tears), meniscus defects (including conditions after meniscectomy), articular cartilage defects, degenerative degeneration of articular cartilage, osteoarthritis (such as knee osteoarthritis, hip osteoarthritis, and shoulder osteoarthritis), traumatic cartilage damage, osteochondritis dissecans, aseptic necrosis, intervertebral disc damage, and temporomandibular joint disorders.

The “meniscus damage” refers to a condition in which the meniscus in the knee joint is damaged, causing various disorders, and specific diseases include meniscus damage (including meniscus damage associated with accidents or sports trauma, or meniscus damage associated with aging), meniscus tears, meniscus degeneration (including sparing meniscus degeneration), and meniscus defects.

The compositions of the present invention (including the pharmaceutical composition) can also be administered before, during, and/or after meniscal suture or meniscal repair.

In the present invention, the “composition” is sufficient if it contains the above-mentioned activin as an active ingredient, and the composition can be used as a pharmaceutical composition for treating or preventing diseases by promoting the proliferation of MSCs, or as a reagent for promoting the proliferation of MSCs. Also, the compositions of the present invention can be used as pharmaceutical compositions for the treatment or prevention of cartilage disorders and for the suppression of degenerative degeneration of cartilaginous tissue through the promotion of MSC proliferation and/or regeneration of cartilaginous tissue. Furthermore, they can also be used as pharmaceutical compositions for relieving pain associated with cartilage disorders (for example, a relieving agent for chronic pain associated with osteoarthritis). Additionally, the compositions of the present invention can also be used as pharmaceutical compositions for treating or preventing meniscus damage.

The content of activin as an active ingredient in the compositions of the present invention can be appropriately adjusted according to the form of activin used, the application of the compositions (such as pharmaceutical composition application and reagent application), and the like, using the aforementioned activity and the like of activin as indicators, and is at least one times the ED50 of activin (such as 0.2 to 1.2 ng/mL), preferably at least 10 or more times the ED50 (such as 2 to 20 ng/mL), more preferably at least 50 or more times the ED50 (such as 10 to 100 ng/mL), further preferably 100 or more times the ED50 (such as 20 to 200 ng/mL), and more preferably 500 or more times the ED50 (such as 100 to 1000 ng/mL). Note that the ED50 of activin is based on the hemoglobin synthesis inducibility using a culture system of human chronic myelogenous leukemia cells (K562 cells) (see Ralph H. Schwall, Cora Lai, Methods in Enzymology, 1991, vol. 198, pp. 340 to 346).

When the composition of the present invention is used as a pharmaceutical composition, the pharmaceutical composition can be formulated by a known pharmaceutical method. For example, it can be used as an injection, gel agent, coater, or the like, usually parenterally, but an injection is preferable in the treatment of knee joints and the like, from the viewpoint of not interfering with fluidity at the site of administration (in the joint, for example). Further, in the treatment of the hip joint and the like, a gel agent (more preferably, a sustained release gel agent) is preferably used because it can be placed in the joint at the time of surgery.

The injection can be prepared by dissolving, suspending, or emulsifying activin or the like in a solvent. The solvent used is, for example, phosphate buffered saline (PBS), physiological saline, sterile water (such as distilled water for injection), culture media (such as MSC culture media), a vegetable oil, an alcohol, and a combination thereof. It can also be produced as a solid agent (such as a freeze-dried product) and dissolved in the above-mentioned solvent before use.

The gel agent can be prepared by mixing activin or the like with a polymer. The polymer used is, for example, hyaluronic acid, collagen (such as type I, II, IV, or XI collagen), gelatin, cellulose, agarose, dextran, xyloglucan, chitosan, chitin, starch, glycosaminoglycans, proteoglycans, fibronectin, laminin, and combinations thereof.

The coater is prepared by triturating or dissolving activin or the like as a base agent. The base agent used is, for example, higher fatty acids or higher fatty acid esters, waxes, surfactants, higher alcohols, silicone oils, hydrocarbons (such as Vaseline and liquid paraffin), glycols, vegetable oils, animal oils, and combinations thereof.

Further, when a polymer substance that is biodegradable and gradually dissolves is contained, it is possible to provide sustained release. Examples of polymer substances include polyvinylpyrrolidone, hydroxypropyl cellulose, hydroxypropyl methyl cellulose, cross-linked sodium carboxymethyl cellulose, carboxymethyl starch, potassium divinyl benzene methacrylate copolymer, and cyclodextrin.

In addition, in these formulations, pharmacologically acceptable carriers may further be combined as appropriate, specifically pain relievers, absorption enhancers, sterile water, physiological saline, PBS, vegetable oils, animal oils, solvents, base agents, emulsifiers, suspensions, surfactants, stabilizers, excipients, vehicles, preservatives, binders, diluents, isotonic agents, bulking agents, disintegrants, buffers, coating agents, lubricants, thickeners, dissolution aids, other additives, and the like.

In addition, the pharmaceutical composition of the present invention may contain other pharmaceutical active components as needed, such as heparin sulfate, hyaluronic acid, chondroitin, glucosamine, collagen, gelatin, steroids, non-steroidal anti-inflammatory drugs, and chondrocyte growth promoters (such as BMP, dexamethasone, TGF-β, FGF, VEGF, HGF, IGF-1, PDGF, CDMP, CSP, EPO, IL, OP, and COP).

Furthermore, the pharmaceutical compositions of the present invention may also take the form of a kit for treating or preventing meniscus damage or cartilage disorders, in combination with the administration devices (such as injectors, gel pipettes, and syringes) described later.

In addition, when the composition of the present invention is used as a reagent, it can be prepared in combination with pharmacologically acceptable carriers as appropriate, specifically media (such as MSC culture media), PBS, physiological saline, sterile water, vegetable oils, animal oils, solvents, base agents, emulsifiers, suspensions, surfactants, stabilizers, vehicles, preservatives, binders, diluents, isotonic agents, buffers, dissolution aids, or other additives, and the like. Note that when the composition of the present invention is used as a reagent for promoting the proliferation of MSCs, it is desirable that it does not contain a chondrocyte growth promoter from the viewpoint of maintaining its differentiation pluripotency.

Products (medicines, reagents) or instruction manuals of the compositions of the present invention can be labeled to indicate that they are used to promote the proliferation of MSCs, to promote the regeneration of cartilaginous tissue, and to treat or prevent meniscus damage or cartilage disorders. Here, “labeled on the product or instruction manual” means labeled on the body, container, packaging, or the like of the product, or labeled on the instruction manual, attached document, promotional material, or other printed material that discloses information about the product.

(Treatment or Prevention Method for Meniscus Damage or Cartilage Disorder)

As shown in the Examples below, Activin A can be used to promote the regeneration of meniscus or cartilage, suppress the degeneration of cartilage, and relieve pain associated with meniscus damage or cartilage disorders. Therefore, the present invention provides a method for treating or preventing meniscus damage or cartilage disorders, including the step of administering activin to the site of the subject's meniscus damage or cartilage disorders.

Meniscus damage or cartilage disorders are as described above. Further, the meniscus damage site or the cartilage disorder site can be understood by those skilled in the art based on the type of the meniscus damage or cartilage disorder disease. In addition, the subject of treatment or prevention of meniscus damage or cartilage disorder can be humans or non-human animals, but is particularly preferably used for humans. In addition, the non-human animals are not particularly limited, and the subject can be various domestic animals, poultry, pets, experimental animals, and the like. Specific examples include, but are not limited to, pigs, cattle, horses (such as racehorses), sheep, goats, chickens, ducks, ostriches, domestic ducks, dogs (such as pet dogs), cats, rabbits, hamsters, mice, rats, and monkeys. Further, not only those who have already suffered from meniscus damage or cartilage disorder, but also those who are likely to develop them, and those who have had or are likely to have recurrent meniscus damage or cartilage disorder can be the subject of the present invention.

The activin administered to the subject may be only the above-mentioned activin, but the form of the above-mentioned pharmaceutical composition is preferably used. When administering activin or a pharmaceutical composition, the dose is appropriately selected according to the age, weight, symptoms, health condition, dosage form of the composition, site of administration, form of administration, and the like (such as injection and application) of the subject, and for human (adult) subjects, activin or a composition containing it, preferably at 0.05 to 100 μg/kg body weight, more preferably at 0.1 to 50 μg/kg body weight, further preferably at 0.5 to 10 μg/kg body weight, and more preferably at 1 to 5 μg/kg body weight (for example, 1.6 μg/kg body weight), is administered once or several times a day. The timing of administration is appropriately selected according to the site of administration, symptoms, and each dosage form, and the like, but examples include the time when the inflammation that occurs after joint invasion has subsided (for example, 7 to 21 days after joint invasion, preferably 10 to 18 days after joint invasion, more preferably 12 to 16 days after joint invasion, and further preferably 14 days after joint invasion). In addition, the method for administration is appropriately selected according to the site of administration, symptoms, and each dosage form, but for example, it can be easily applied to the affected area (meniscus damage site or cartilage disorder site) by using an injector, gel pipette, or syringe.

Furthermore, as described above, in the present invention, other pharmaceutical active components may be administered in combination with activin. In addition, from the viewpoint of facilitating more effective treatment of meniscus damage and cartilage disorders, it is desirable to administer MSCs as well (as to the method for treating cartilage disorders using MSCs, see International Application Japanese-Phase Publication No. 2010-501547, Japanese Unexamined Patent Application Publication No. 2014-196354, and the like). The MSCs are as described above, but it is preferable to use synovium-derived MSCs because they have a higher cartilage-forming ability. Note that in the present invention, administration “in combination” includes not only administration at the same time as activin, but also administration of other pharmaceutical active components and/or MSCs before or after administration of activin.

(Method for Predicting Prognosis of Cartilage Disorders)

As shown in the Examples described later, it was clarified that patients with higher intra-articular concentrations of Activin A had a better prognosis for cartilage disorders. Therefore, the present invention provides a method for predicting prognosis of a cartilage disorder in a joint, comprising the steps of: measuring a concentration of activin in a joint of a subject; and predicting that the prognosis of the cartilage disorder in the subject is good if the concentration obtained in the step is higher than a reference value.

The subject and cartilage disorder are as described above. The “measurement” of activin concentration in a joint of the subject is usually performed on the synovial fluid in the joint. The “time of measurement” is not particularly limited, but as shown in the Examples below, the concentration of activin tends to increase during the acute inflammatory phase, and this phase is preferable from the viewpoint that it is easier to detect, and more specifically, it is after joint invasion (for example, after joint surgery), more preferably the first day to one week after joint invasion, and more preferably the third to fourth day after joint invasion. The “measurement method” is not particularly limited either, but a detection method (immunological method) using an antibody that specifically binds to activin is usually used. More specifically, immunoassays (enzyme-linked immunosorbent assay (ELISA, EIA), fluorescence assay, radioimmunoassay (RIA), immunochromatography, and Western blotting can be mentioned. Further, a method using a detection device using a surface plasmon resonance phenomenon (for example, BIAcore (manufactured by GE Healthcare)) using a metal thin film on which a compound specifically bound to activin is immobilized can also be mentioned.

The “reference value” in the present invention is a value (such as a cutoff value) that serves as an indicator in determining the prognosis of cartilage disorders, and specifically, the reference value is a concentration of activin in the synovial fluid of 550 pg/mL, preferably 750 pg/mL, and more preferably 950 pg/mL. Then, in the method of the present invention, if the measured value is significantly higher than that reference value, it is predicted that the prognosis of cartilage disorders (such as after surgery) tends to be good, while if it is lower, it is predicted that the prognosis tends to be poor.

In addition, if the prognosis is predicted to be poor as above, supplementing the subject with activin (for example, activin is supplemented until the concentration reaches the reference value) may improve the cartilage regenerative capacity in the joint of the subject and improve the prognosis.

Thus, the present invention also provides a method for treating or preventing cartilage disorders, including the steps of: measuring the concentration of activin in the joint of the subject; and administering activin, or activin and MSCs, to the subject's cartilage disorder site if the concentration obtained in the step is lower than the reference value.

### EXAMPLES

Hereinafter, the present invention is described in more detail based on Examples, but the present invention is not limited to the following Examples. In addition, the Examples were carried out using the following materials and methods.

(Material)

Recombinant human IL1b, IL6, IL8, PDGF-AA, and Activin A were purchased from R&D Systems Inc. (Minneapolis, Minn., USA). MEM-alpha (modified Eagle's medium), FBS (fetal bovine serum), antibiotic-antifungal solution (hereafter referred to as “antibiotic”), and trypsin-EDTA solution were purchased from Gibco BRL (MA, USA). Collagenase D was purchased from Sigma-Aldrich (MO, USA). MTT (3-(4,5-dimethyl-2-thiazolyl)-2,5-diphenyltetrazolium bromide) was purchased from Dojindo Molecular Technologies, Inc. (Kumamoto, Japan).

(Ethics)

All human-related studies were approved by the Ethics Committee of Tokyo Medical and Dental University (approval numbers: No. 455 and No. 2121). Full written informed consent to participate in the present study was obtained from all patients involved in the study. All animal-related studies were approved by the Animal Experiment Committee of Tokyo Medical and Dental University (approval number: No. 0160274). All animal experiments were conducted in accordance with the guidelines of our organization.

(Preparation of Synovial Fluid for Arthrocentesis and Protein Analysis)

Synovial fluid was collected by arthrocentesis immediately before and 3 to 4 days after anterior cruciate ligament reconstruction (ACL-R, see Inoue M, et al. J Exp Orthop. 2016 December; 3(1): 30).

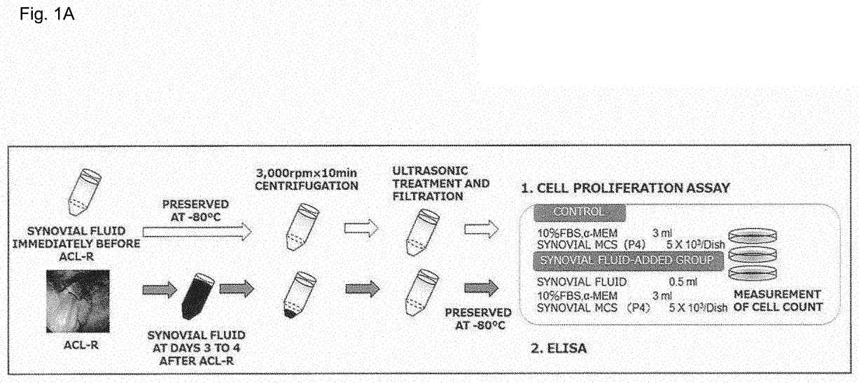

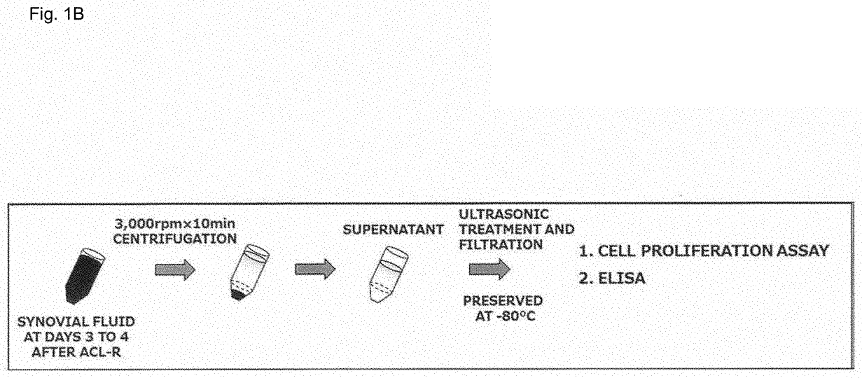

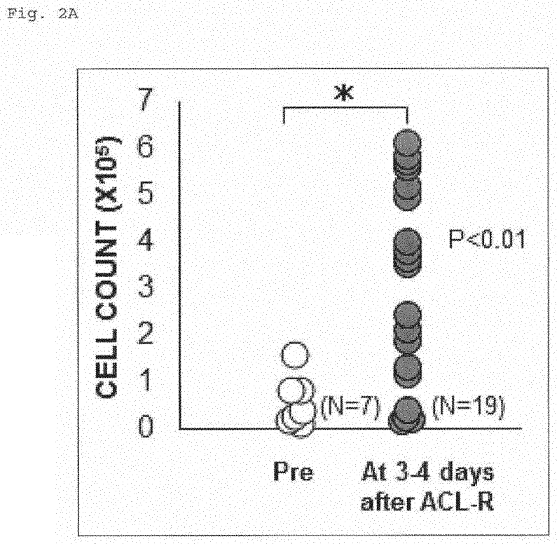

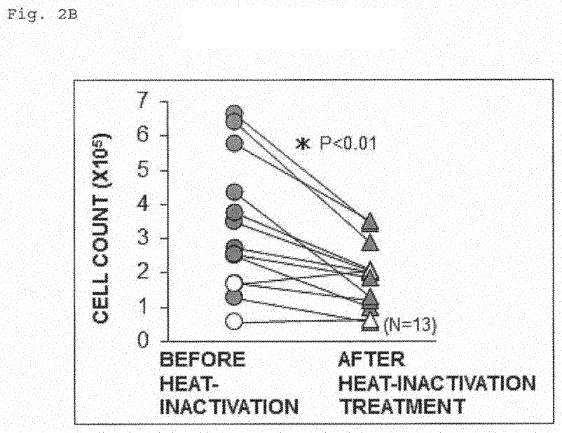

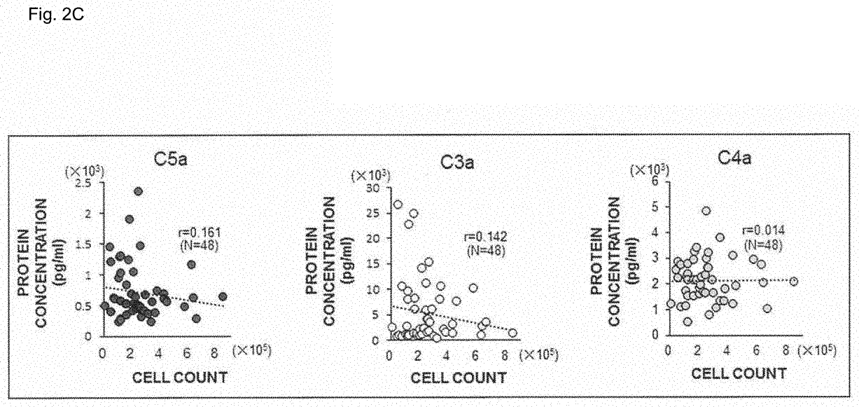

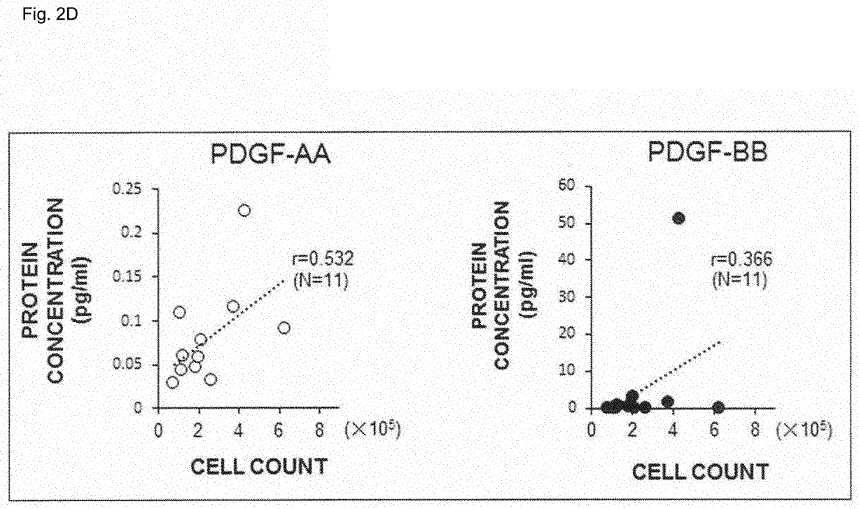

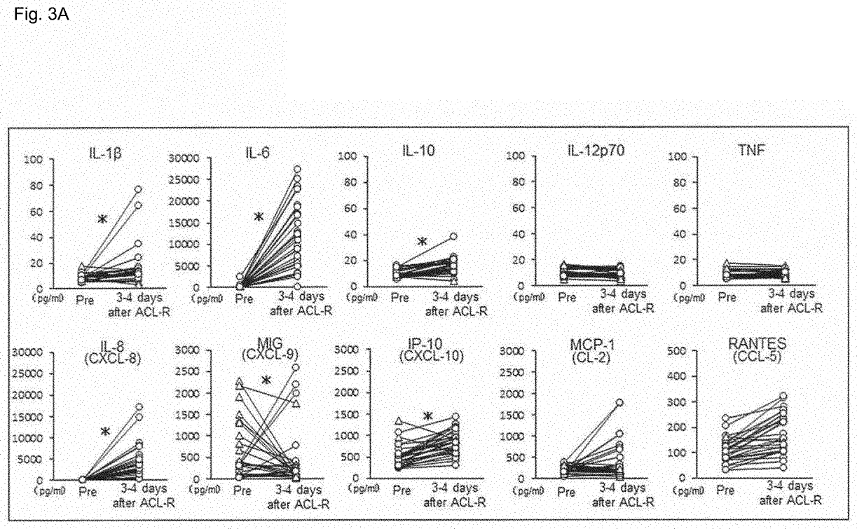

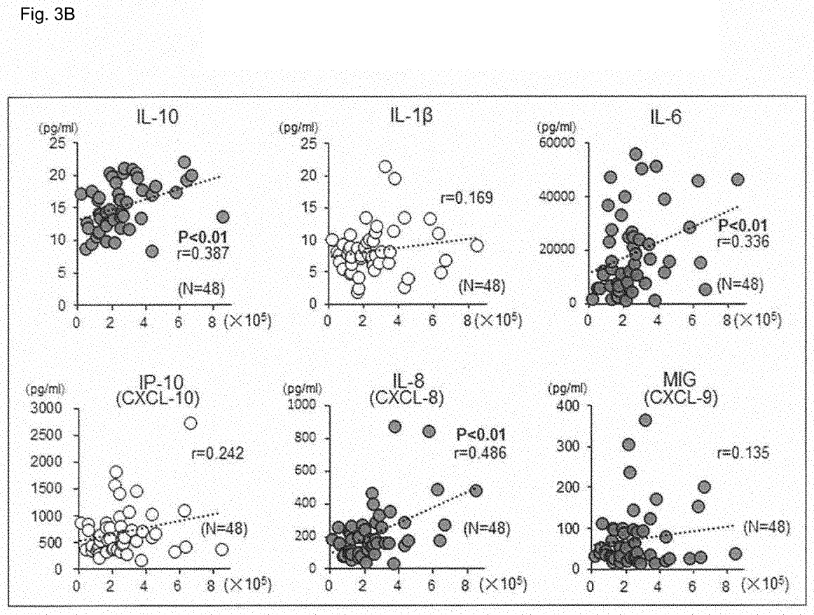

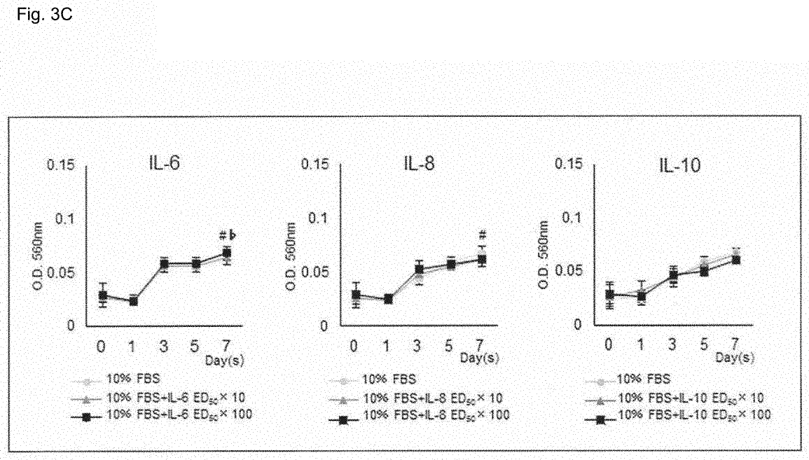

To perform initial evaluation of pro-proliferative activity and cytokine/chemokine concentrations in the synovial fluid collected immediately before and 3 to 4 days after ACL-R (see FIG. 2A and FIG. 3A), debris was removed from the cryopreserved synovial fluid by centrifugation, and then the synovial fluid was sonicated (Amplitude Settings at 40%, Q125 Sonicator, QSONICA LLC., Newton, Conn., USA) to reduce its viscosity for the experiment (see FIG. 1A).

In addition, in the experiments to identify bioactive substances from synovial fluid (see FIGS. 2B-D, 3B, and 4A-C), the synovial fluid was immediately subjected to centrifugation at 860×g for 10 minutes (centrifugation at 2,000 rpm for 10 min, Becton Dickinson, Franklin Lakes, N.J.) after collection to remove the cellular fraction in order to more strictly exclude the influence of substances derived from cellular components contained in the synovial fluid. Then, in order to reduce the viscosity, the supernatant was ultrasonically treated for 10 seconds and passed through a 70 μm nylon mesh to remove debris (see FIG. 1B). After that, the cryopreserved synovial fluid was used in the experiments.

Note that although not shown in the figure, cytokine/chemokine levels in the synovial fluid were analyzed and tested, and among those tested, only the IL-8 levels were significantly reduced in the synovial fluid that had been centrifuged prior to cryopreservation to exclude cellular components. In addition, information on the patients who participated in this study is shown in Table 1.

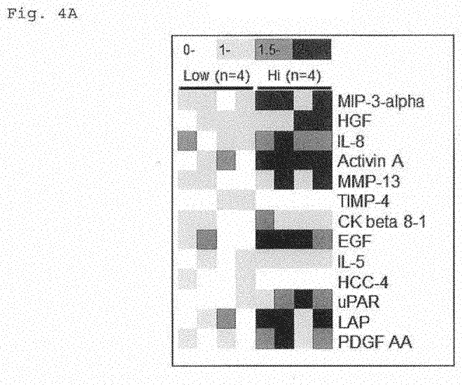

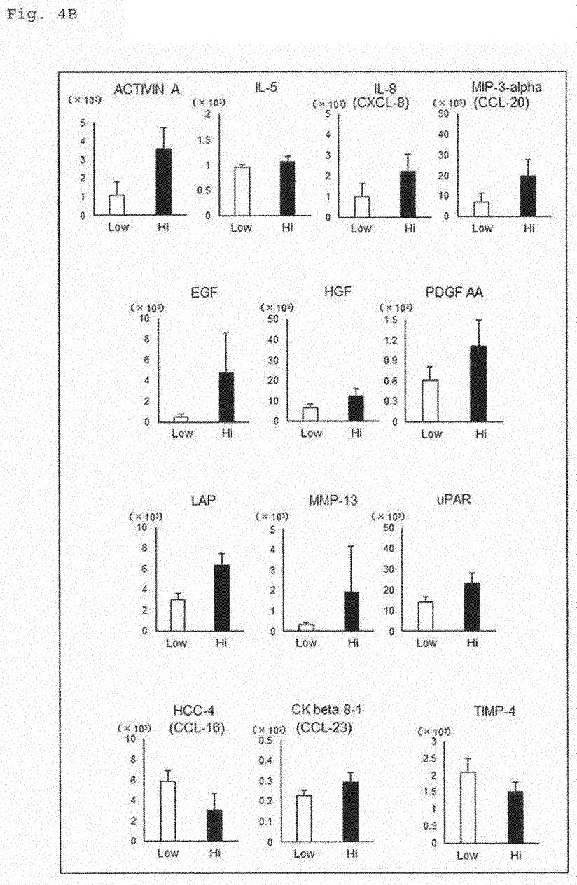

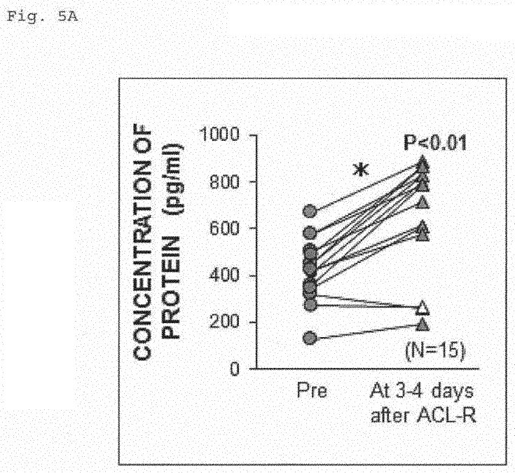

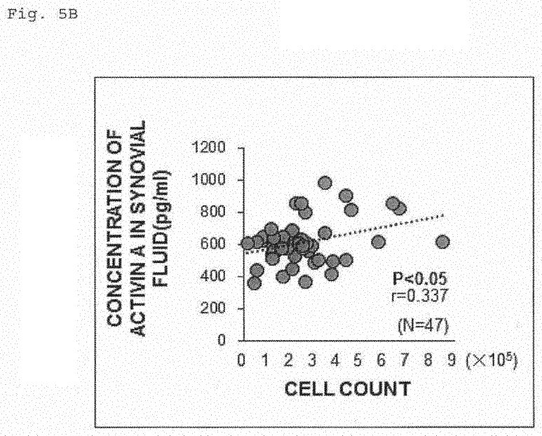

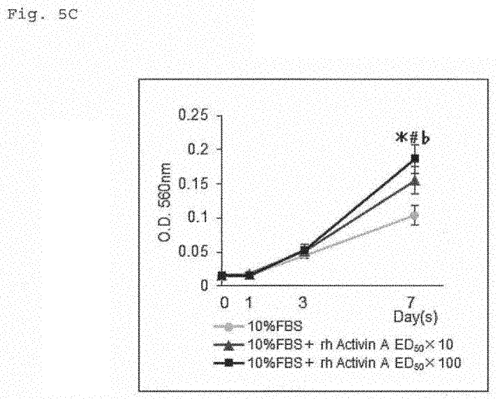

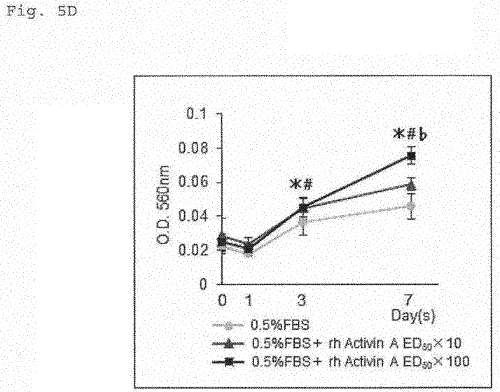

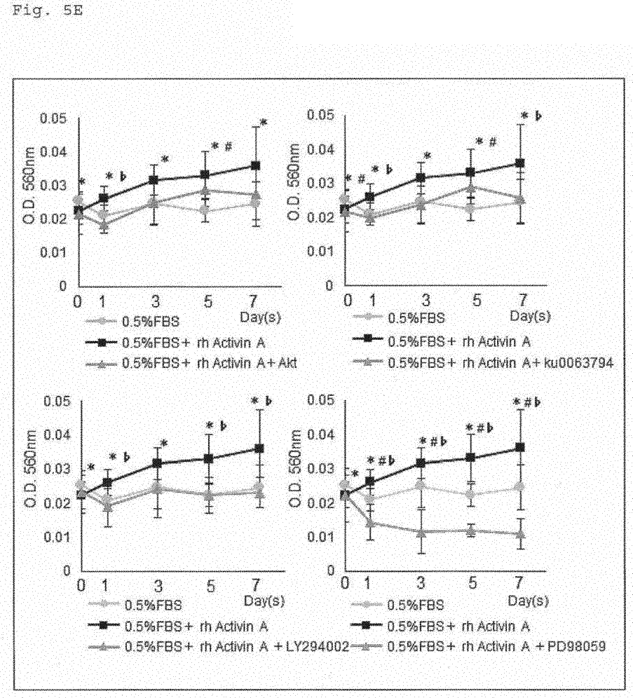

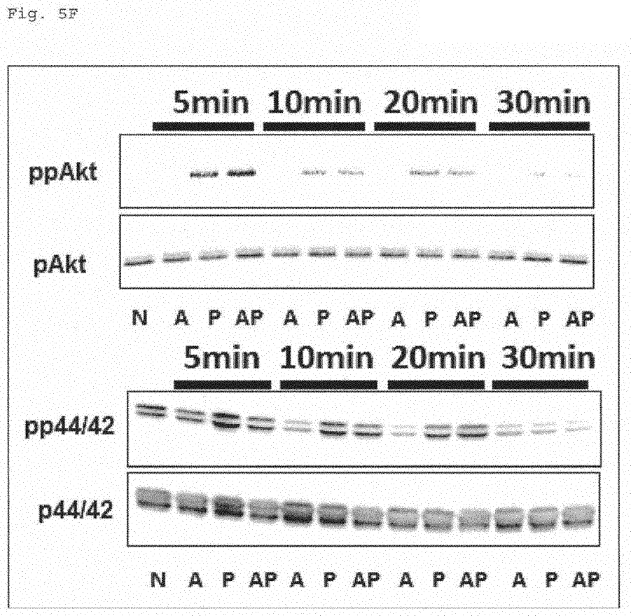

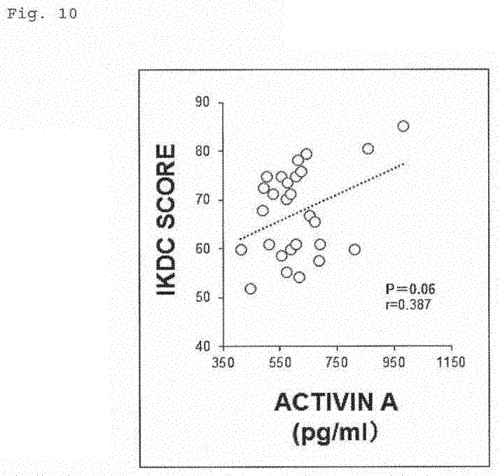

TABLE 1

| | | | Number | Gender | | |
|---|---|---|---|---|---|---|
| | Corresponding | Pre/After | of | (Male/ | Age | |
| Experimental Item | Figure | [symbol] , ect | Patients | Female) | Median | Range |
| Collection of | Fig. 2A | Pre | 7 | 2/5 | 32.7 | 13-46 |
| synovial fluid | | | | | | |
| from patients pre | | After | 19 | 7/12 | 36.5 | 12-62 |
| and after ACL-R | | | | | | |
| Heat-inactivation | Fig. 2B | After | 13 | 4/9 | 31.7 | 17-52 |
| treatment of | | | | | | |
| syncvial fluid | | | | | | |
| Correlation anaylsis | Fige. 2C | After 48 | 48 | 22/26 | 23.5 | 12-59 |
| between cytokines | and 3B | | | | | |
| and chemokines, | | | | | | |
| and their pro- | | | | | | |
| proliferative activities | | | | | | |
| Correlation analysis | Fig. 2D | After | 11 | 6/8 | 22 | 15-30 |
| between [symbol] and [symbol] , | | | | | | |
| and their pro- | | | | | | |
| proliferative activities | | | | | | |
| [symbol] bead array | Fig. 3A | Pre/After | 28 | 13/15 | 24.6 | 12-53 |
| <CBA> analysis | | | | | | |
| Antibody-based | Figs. 4A | Pro-proliferative | 4 | 0/2 | 21.3 | 16-33 |
| protein array | | activity: Low | | | | |
| | and 4B | Pro-proliferative | 4 | 0/4 | 22.3 | 15-28 |
| | | activity: [symbol] | | | | |
| Quantification | Fig. 5A | Pre/After | 15 | 7/8 | 27.9 | 12-58 |
| of Activin & in | | | | | | |
| synovial fluid | | | | | | |
| Correlation analysis | Fig. 5B | After | 47 | 22/25 | 25.7 | 12-59 |
| between Activin A | | | | | | |
| and pro- | | | | | | |
| proliferative activity | | | | | | |
| Correlation analysis | Fig. 10 | After | 27 | 14/13 | 26 | 12-59 |
| between Activin A | | | | | | |
| and IRDC score | | | | | | |

[symbol]

indicates data missing or illegible when filed

(Isolation and Expansion Culture of Synovial Mesenchymal Stem/Stromal Cell)

Primary human synovial cells were isolated from the synovial tissues obtained from patients who underwent total knee arthroplasty (TKA), as described in Sekiya I. et al, Clin Orthop Relat Res, 2015, 473(7), pp. 2316 to 2326. That is, first, 0.5 to 1 mg of synovium obtained by incising the suprapatellar bursa was subjected to collagenase D treatment (Sigma, MO, USA, 3 hours at 37° C.) to disperse nucleated cells. Debris was removed by passing the cell dispersion through a 70 μm nylon mesh. Nucleated cells were seeded in a 15 cm dish at 5.7×10² cells/cm² and maintained and cultured in MEM-alpha medium supplemented with 10% FBS and antibiotics (Gibco, MA, USA) for 14 days. The cells were then separated from the dish in TripLE solution (Gibco) and seeded twice more in a new dish to obtain a sufficient cell count to be used in the experiments. Note that in all experiments, cells within 6 passages were used.

(Evaluation of Pro-Proliferative Activity of Synovial Fluid)

To evaluate the pro-proliferative activity of synovial fluid (see FIGS. 2A-D, 3A, 3B, and 5B), synovium-derived MSCs were seeded in 6 cm dishes to reach a cell count of 5×10³, and maintained and cultured in normal growth medium supplemented with 14% synovial fluid (MEM-alpha, 10% FBS and antibiotics) at 37° C. for 2 weeks. Then, the cells were separated from the dishes with trypsin/EDTA, and the cell count was measured with an automatic cell count measuring device (TC-20; Bio-Rad, Hercules, Calif., USA). In addition, dead cells were removed by trypan blue staining.

(Quantification of Cytokines, Complement, and Proliferation Factors in Synovial Fluid)

The levels of cytokines/chemokines and complement in the synovial fluid were measured using Cytometric Bead Array System (CBA Kit, BD Biosciences, San Jose, Calif., USA) according to its use instruction manual. In addition, to detect signals, FACS Verse Flow Cytometry (BD Bioscience), data analysis software, and BD FCAP Array Software (BD Biosciences) were used. To quantify human IL8 (CXCL8), IL1b, IL6, IL10, TNFa, and IL12, the human inflammatory cytokine CBA kit was used. In addition, the human chemokine CBA kit was used to analyze human MIG (CXCL9), IP10 (CXCL10), MCPJ (CCL2), and RANTES (CCL5). The human anaphylatoxin kit was used to quantify human C3a, C4a, and C5a. To determine the levels of PDGF-AA, PDGF-BB, and Activin A in the synovial fluid, ELISA (enzyme-linked immunosorbent assay) was performed using an ELISA kit (Human Quantikine ELISA systems, R&D systems, Minneapolis, Minn., USA) according to its use instruction manual.

(Proliferation Assay of Synovial MSCs with Recombinant Protein)

To evaluate the pro-proliferative activity of recombinant proteins (IL6, IL8, IL10, and Activin A), MTT assays were performed (see FIGS. 3C and 5C-E). Synovial MSCs were seeded to 1×10³ cells/well in 96-well plates and cultured in 0.2 mL of normal growth medium 1 day before being subjected to recombinant protein treatment. On the following day, the medium was replaced with a fresh one (normal growth medium or medium with a reduced FBS concentration of 0.5% and recombinant protein added (10 times the concentration of ED50 or 100 times the concentration of ED50)). MTT assays were performed at days 0, 1, 3, 5, and 7 according to the method described in Denizot F. et al., J Immunol Methods, 1986, 89(2), pp. 271 to 277.

(Protein Array with Antibody)

A differential expression assay was performed to identify novel bioactive compounds present in synovial fluid and significantly promote the proliferation of synovial MSCs. Glass Chip-Based Human Cytokine Antibody Array System G (RayBio Series G-2000 Series, slides AAH-CYT-G2000-8, RayBiotech Inc. Norcross, Ga., USA) was used to quantify the expression levels of 174 proteins in synovial fluid according to its use instruction manual. Further, in synovial fluids with a high pro-proliferative activity (n=4) and synovial fluids with a low one (n=4), proteins that were differentially expressed were analyzed. Then, a total of 13 proteins were selected as those with statistically different expression levels.

(Western Blotting)

Synovial MSCs were seeded in 6 cm dishes to reach a cell count of 2×10⁵, and cultured in normal growth medium for 1 day. The medium was replaced with fresh medium with reduced amount of FBS (MEM-alpha, 0.5% FBS and antibiotics) and cultured for one more day. The cells were treated with rh Activin A and/or PDGF-AA (amount 3 times the ED50) for up to 30 minutes. Then, those cells were lysed with cell lysis buffer (#9803S; Cell Signaling Technology, MA) to prepare total cell lysate.

Western blotting was performed as described in Uomizu, M. et al, J Med Dent Sci. vol. 65, pp. 73 to 82. The antibodies used were purchased from Cell Signaling Technology (phospho-p44/42 (#4370), p44/42 (#9102), phospho-Akt (#9271), and Akt (#9272)).

(In-Vitro Differentiation Assay)

In-vitro differentiation assays were performed according to the method described in Colter D C. et al, Proc Natl Acad Sci USA, 2001, 98, pp. 7841 to 7845.

To form chondrogenic spheroids, 2.5×10⁵ cells were transferred into 15 mL polypropylene tubes (BD Biosciences) and subjected to centrifugation at 450×g for 10 minutes. Then, the cell pellets were cultured in chondrogenic medium containing 1000 ng/mL rhBMP-2 (Infuse Bone Graft; Medtronic, TN, USA), 10 ng/mL transforming proliferation factor-133, and 100 nM dexamethasone (Sigma-Aldrich, MO, USA) for 14 days.

For calcification, cells were cultured in normal growth medium supplemented with 1 nM dexamethasone, 20 mM β-glycerophosphate, and 50 μg/mL ascorbic acid-2-phosphate (Sigma-Aldrich) for 21 days. Calcified nodule formation was visualized by alizarin red staining (Sigma-Aldrich).

For adipogenesis, the cells were cultured for 21 days in MEM-alpha supplemented with 10% FBS, 100 nM dexamethasone (Sigma-Aldrich), 0.5 mM IBMX (isobutyl-1-methylxanthine; Sigma-Aldrich), and 50 μM indomethacin (Wako, Tokyo, Japan). The adipogenic cultures were immobilized and stained with Oil Red O solution (Sigma-Aldrich).

(Expression Analysis of Surface Antigens)

Cells were isolated by treatment with TrypLE (Thermo Fisher Scientific, MA, USA) for 5 minutes and stained with antibodies as described in Tsuji K. et al, Cell Transplant. 2017, 26(6), pp. 1089 to 1102. The surface antigen positive cell fraction was then measured using FACS Verse Flow Cytometer (BD Biosciences). Note that fluorochrome-conjugated antibodies for the analysis of cell surface markers (CD73, CD90, CD105, and CD44) were purchased from BD Biosciences (NJ).

(Resection of Anterior Half of Medial Meniscus)

Twenty male C57BL/6J mice (8 weeks old, weight: about 25 g) were used for this study (5 mice in each group. ORIENTAL YEAST Co., Ltd. Tokyo, Japan). After anesthesia by isoflurane inhalation, a straight incision was made in the left anterior knee, and the anterior central side of the joint capsule was cut. The patellar tendon was then dislocated laterally to expose the anterior corner of the medial meniscus (see Hiyama K. et al, J Orthop Res., 2017, 35(9), pp. 1958 to 1965). The anterior corner of the meniscus was cut and dislocated anteriorly using forceps. The medial meniscus was cut perpendicularly at the medial collateral ligament, and the anterior portion of the medial meniscus was resected in a microscopic operation using a surgical microscope. The skin was closed in layers with non-absorbable sutures. Note that the right knee was subjected to sham surgery. In addition, the mice were kept under a 12-hour light/dark cycle with free movement, free feeding, and drinking water.

Recombinant human Activin A (42 ng/6 μL in PBS) and/or rh PDGF-AA (174 ng/6 μL in PBS) was injected intra-articularly on postoperative day 7 (see Hoshino T. et al., BMC Musculoskelet Disord., 2018, 19(1): 291). The mice were euthanized by carbon dioxide on postoperative day 10 or 21, and the left knee was dissected.

(Histology and Immunohistology)

Tissue samples were fixed in 4% paraformaldehyde for 3 to 4 days and subjected to decalcification in 20% ethylenediaminetetraacetic acid (EDTA) solution for 21 days. Then, they were embedded in paraffin wax. The specimens were then cut to a 5 μm thickness in the sagittal plane and stained with safranin O/Fast Green or hematoxylin/eosin (H&E) to histologically evaluate meniscus regeneration. The histological sections were observed with an Olympus BX53 microscope (Olympus, Tokyo, Japan).

A modified Pauli's score system was used to histologically quantify meniscus regeneration (see Hiyama K. et al, J Orthop Res. 2017, 35(9), pp. 1958 to 1965). Histological evaluation was performed by two investigators in a blind manner.

Type I and II collagen immunostaining was performed as described in Tsuji K. et al, Cell Transplant., 2017, 26(6), pp. 1089 to 1102. That is, sections were subjected to deparaffinization with xylene (Wako Pure Chemical Industries, Tokyo, Japan), rehydrated stepwise with alcohol, and saturated with phosphate buffered saline (PBS). For type II collagen staining, the samples were pretreated with 50 mM Tris-HCl buffer (Wako Pure Chemical Industries) containing 0.4 mg/ml Proteinase K (Dako, Agilent Technologies, Santa Clara, Calif., USA) for 10 min. For type I collagen staining, no digestion with Proteinase K was performed.

Next, the cells were treated with methanol containing 0.3% hydrogen peroxide for 30 minutes to suppress the activity of endogenous peroxidase. As primary antibodies, anti-type I collagen antibody (1:200 dilution; Abcam cat. #ab34712, Cambridge, UK) or anti-type II collagen antibody (1:1000 dilution; Abcam cat. #ab34712, Cambridge, UK) were added to the sections and incubated at 4° C. overnight. Then, the sections were rinsed with 0.1% PBS-Triton X-100 (MP Biomedicals Inc., Solon, Ohio, USA) and incubated with secondary antibodies diluted 1:200 [Biotinylated goat-derived anti-rabbit immunoglobulin G (IgG), Vector Laboratories, Burlingame, Calif., USA]. Then, the sections were rinsed, the signals were visualized with Vectastain ABC Reagent (Vector Laboratories), and diaminobenzidine staining (DAB, Vector Laboratories) was applied. In addition, the sections were contrast stained with hematoxylin (Muto Pure Chemicals, Tokyo, Japan).

(MIA-Induced Arthritis Rat Model)

Wistar rats (Charles River, Japan) aged 10 weeks and weighing 330 to 345 g were used for this study. The rats were divided into two groups (those to be injected with Activin A after injection of mono-iodoacetic acid (MIA), and those to be injected with vehicle after injection of MIA (control)), and subjected to the following experiments.

First, the rats were anesthetized by isoflurane inhalation, and 30 μL of physiological saline containing 1.0 mg MIA was injected into the left knee joint as described in Hoshino T. et al, BMC Musculoskelet Disord., 2018, 19(1): 291. Then, at day 14 after MIA injection, 30 μL of recombinant human Activin A (400 ng/30 μL in PBS) or PBS only as a vehicle was injected. Then, pain avoidance behavior (load asymmetry) between the left limb (MIA-injected side) and the right limb (MIA-uninjected side) was evaluated at day 0 (before injection) and days 1, 3, 7, 14, 17, 19, 21, 24, and 28 after injection.

Load asymmetry was detected by using an incapacitance tester (Linton Instrumentation, Norfolk, UK) with rats in its prismatic Plexiglas case, and the left and right hindlimbs placed on independent force plates. Each loading was measured 100 times, and the change in the percentage (%) of weight applied to the left knee (MIA-administered side) hindlimb was calculated based on the following formula (see Hoshino T. et al., BMC Musculoskelet Disord. 2018, 19(1): 291, Inomata K. et al., BMC Musculoskelet Disord. 2019, 20(1): 8, and Onuma H et al., J Orthop Res. 2020, Jan. 5. doi:10.1002/jor.24580).

Percentage (%) of load on the left knee hindlimb=Load on the left knee hindlimb/(Load on the left knee hindlimb+Load on the right knee hindlimb)×100

Then, the change from the difference in the percentage of load before administration was then evaluated over time.

(Statistical Analysis)

All statistical analyses were performed using SPSS software. The Mann-Whitney's U-test and Student T-test were used for the statistical analyses. When the P-value was less than 0.05, it was determined that there was a significant difference.

### (Example 1) Identification of Substances with MSC Pro-Proliferative Activity

As shown in FIGS. 1A and B, synovial fluid collected from anterior cruciate ligament reconstruction (ACL-R) patients was added to the MSC culture system at a ratio of 14%, and cell proliferative activity was measured. As a result, as shown in FIG. 2A, it became clear that there were bioactive substances that activated the cell proliferation of MSCs in the synovial fluid, although there were very large individual differences. In addition, the pro-proliferative activity in the synovial fluid collected from patients immediately before ACL-R was slight. Therefore, these substances appear to be particularly increased in synovial fluid after invasion (ACL-R) (for example, 3 to 4 days after ACL-R, acute inflammatory phase).

Based on the results in FIG. 2A, analysis was carried out on the bioactive substances that was present in the synovial fluid and supported the activation of MSC proliferation in vitro. As a result, heat-inactivation of the synovial fluid (inactivation by heating at 56° C. for 30 minutes) significantly reduced the cell proliferation-promoting effect, suggesting that the molecules of interest were thermosensitive and complement (see FIG. 2B). However, as shown in FIG. 2C, no positive correlation was observed between the concentrations of complement proteins (C3a, C4a, and C5a) and cell proliferative activity in the synovial fluid. Therefore, the conclusion was that these molecules were not the target ones.

Analysis was also carried out on PDGFAA and PDGFBB, which are present in serum and have already been identified as proliferation factors of synovial MSCs. As a result, as shown in FIG. 2D, the PDGF level in the synovial fluid was positively correlated with the MSC proliferative activity of the synovial fluid, indicating that these molecules could be candidates for the bioactive substances sought.

In addition, correlation analysis was carried out with various cytokines and chemokines whose concentrations were thought to be increased by joint invasion. As a result, as shown in FIG. 3A, it became clear that IL1β, IL6, IL10, IL8, MIG, and IP-10 were increased in expression level by joint invasion. Furthermore, as shown in FIG. 3B, there was a significant positive correlation between the concentrations of IL1β, IL6, and IL8 in the synovial fluid and the ability to activate MSC proliferation among the molecules examined. However, as a result of culturing MSCs by adding the recombinant proteins IL6, IL8, and IL10 to the respective normal growth media, no proliferation activation of MSCs was observed even when the recombinant proteins of the cytokines and chemokines used were added to the media to 100 times the ED50, as shown in FIG. 3C. In other words, there was no target among IL1β, IL6, IL10, IL12, TNFa, CXCL8, CXCL9, CXCL10, CL2, and CCL5.

In view of the above, the patients were divided into two groups based on the proliferative activity of the synovial fluid, and analysis was carried out on the proteins with different abundances using an antibody array (Raybiotech, Cytokine Array) capable of quantitatively analyzing 174 proteins simultaneously.

As a result, there were 13 types of molecules that were determined to have significant differences in expression levels between the two groups by U-test, as shown in FIG. 4A-B. One of the most interesting molecules (Activin A) was further analyzed as follows.

<Evaluation of MSC Proliferation Promotion and the Like by Activin>

As shown in FIG. 5A, a significant increase in the concentration of Activin A was observed in the synovial fluid after the invasion. In addition, as shown in FIG. 5B, there was a significant positive correlation between the concentration in the synovial fluid and the cell proliferative activity of the synovial fluid, suggesting that activin functioned as a proliferation factor for MSCs.

Next, to analyze the bioactivity of activin in more detail, the Activin A recombinant protein (rh Activin A) was added to the normal growth medium of MSCs, and as shown in FIGS. 5C and D, the proliferation of MSCs was significantly activated, independent of serum concentration. This suggests that the presence of cofactor is not essential for the activation of proliferation by activin.

In addition, the present inventors tried to clarify the molecular mechanism of MSC proliferative activity by Activin A. It has been reported that the ERK and PI3K signal transduction pathways are activated for MSC proliferation (see Uomizu, M. et al, J Med Dent Sci., 2018, vol. 65, pp. 73 to 82). With the above in mind, Activin A (rh Activin A) was applied to synovial MSCs in the presence of PI3K-PKB/Akt and Erk1/Erk2, a specific inhibitor for each of these signal transduction pathways, and the cell count was measured by MTT assay.

As a result, as shown in FIG. 5E, the pro-proliferative activity of Activin A was completely eliminated when the inhibitor of PI3K-Akt pathway, LY294002, Akt1/2, or KU0063794, was added. On the other hand, it also became clear that when the Erk1/Erk2 inhibitor PD98059 was added, the cell count was further reduced than without the addition of Activin A. These results suggest that the pro-proliferative activity of Activin A requires the activation of PI3K-PKB/Akt and Erk1/2.

Furthermore, analysis was carried out on the early phosphorylation of intracellular proteins in MSCs that were serum-starved for 24 hours, and as shown in FIG. 5F, phosphorylation of Akt and Erk1/2 was not detected within 30 minutes after Activin A treatment. However, it became clear that Activin A enhanced the phosphorylation of Akt by PDGF. In contrast, no enhancement of intracellular Erk phosphorylation was observed.

The above suggests that activin acts additively on the Pl3K-Akt/PKB signal transduction pathway to induce activation of cell proliferation.

### (Example 2) Analysis of Effect of Activin on Undifferentiation of MSCs

To evaluate the effect of activin on the stemness of MSCs, they were cultured for 2 weeks in the presence of rh Activin A, and changes in the expression level of MSC surface antigens were examined by flow cytometric analysis.

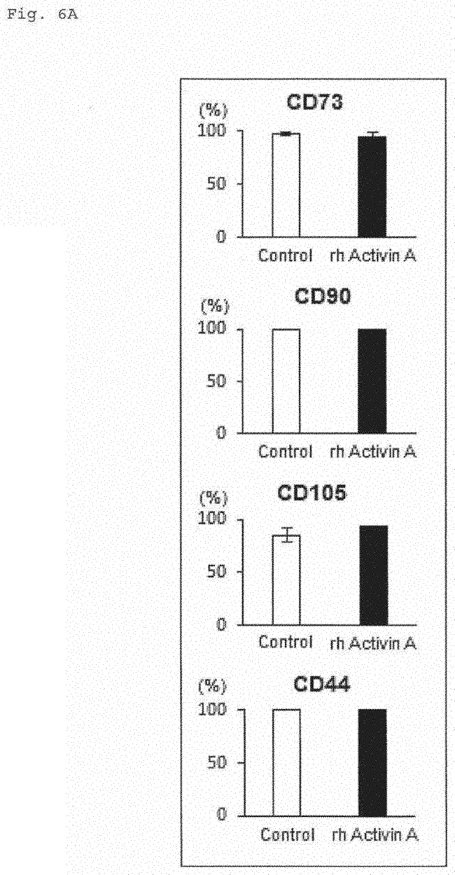

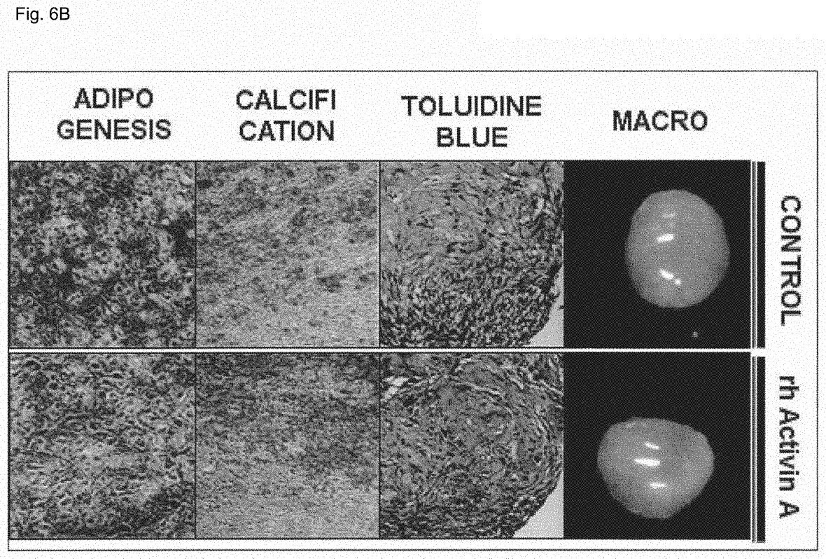

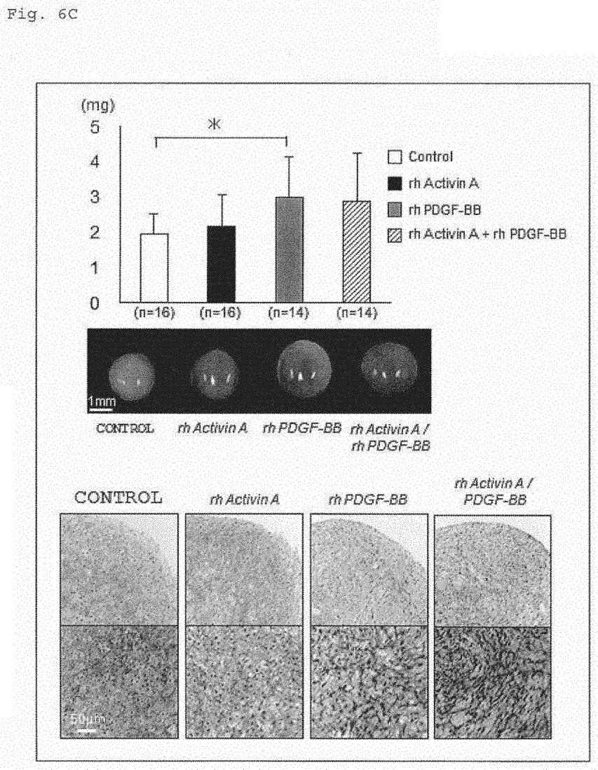

As a result, as shown in FIG. 6A, no change in the cell population positive for MSC-related antigens by Activin A was observed. This suggests that activin activates proliferation without altering the phenotype of MSCs in in-vitro culture systems.

In addition, after 2 weeks of culture in the presence of Activin A, differentiation induction in the directions of chondrocytic-, osteoblastic-, and adipocytic-cells in vitro was performed, and as shown in FIG. 6B, no significant changes were observed compared to the control (cells cultured without the addition of Activin A). Thus, no change was observed in the differentiation induction in the directions of cartilage, bone, and adipocytes, suggesting that, together with the results shown in FIG. 6A above, activin activated proliferation of MSCs without compromising their stemness.

Furthermore, when Activin A and the like were added to the chondrogenic differentiation medium, as shown in FIG. 6C, the addition of PDGF was also observed to have an effect of promoting the formation of chondrocyte aggregates (increase wet weight), but no significant promotive effect of activin on the production of cartilage matrix was observed.

The above analysis results have clarified that activin is a molecule that has a cell proliferation-promoting effect in vitro without compromising the stemness of MSCs.

### (Example 3) Medial Meniscus Anterior ½ Resection Model

Next, tissue regeneration experiments were carried out to investigate the physiological effects of activin in vivo. Based on the above in-vitro results showing that Activin A promotes the proliferation of MSCs, first, examination was carried out on the effect of Activin A (rh Activin A) administration on a regeneration model after tissue defect (medial meniscus anterior ½ resection model).

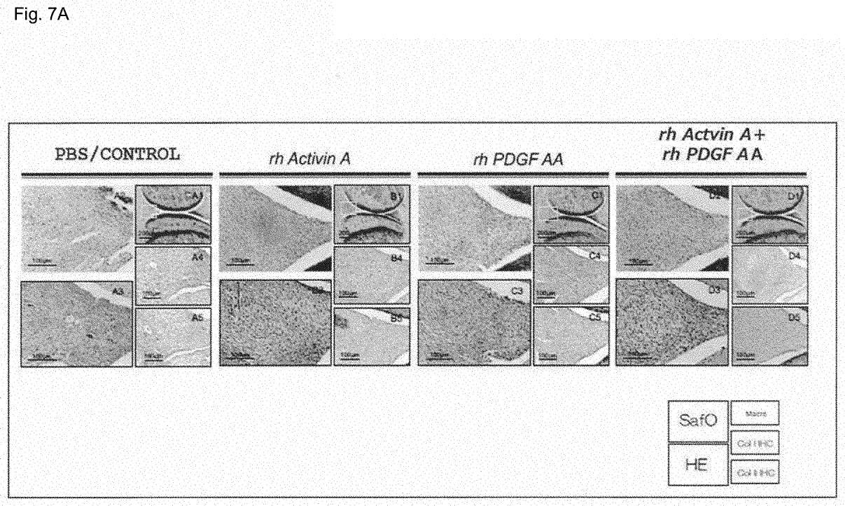

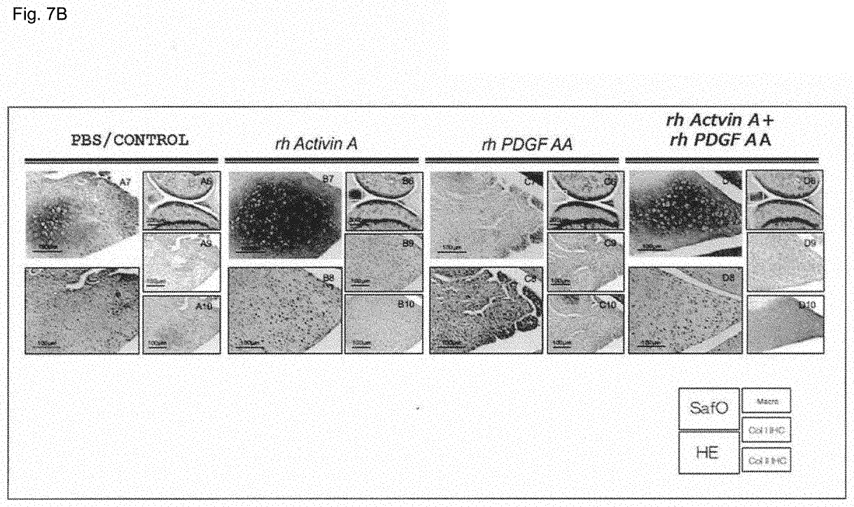

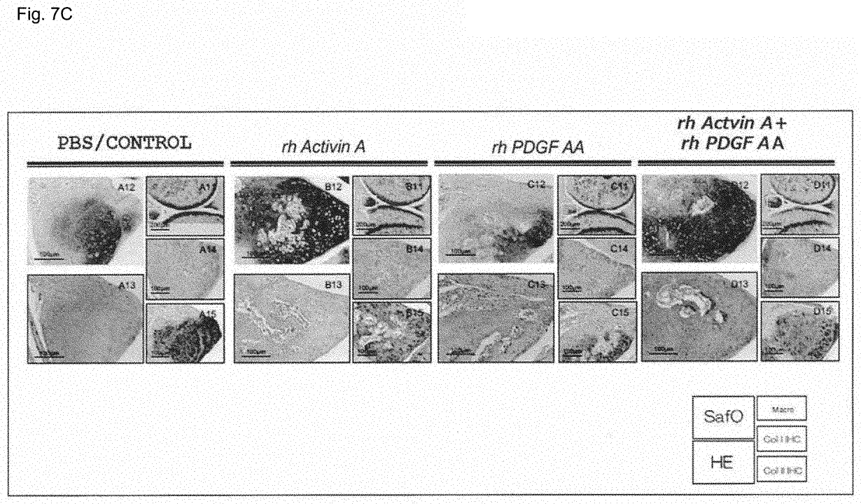

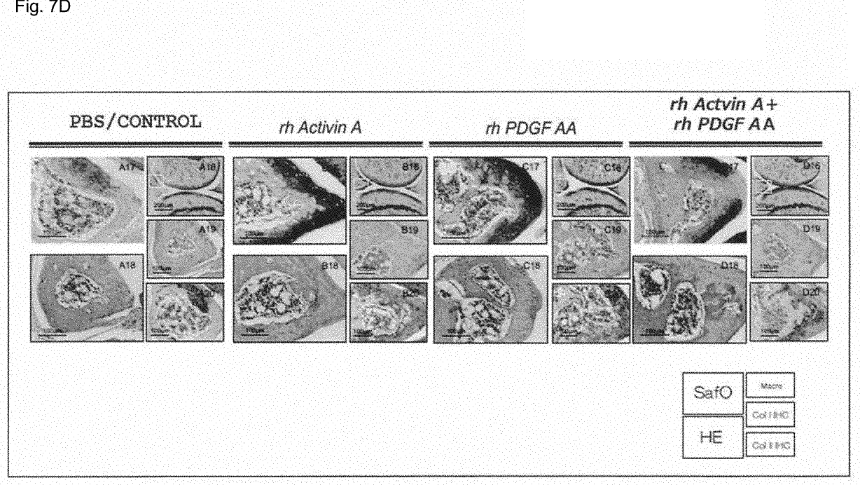

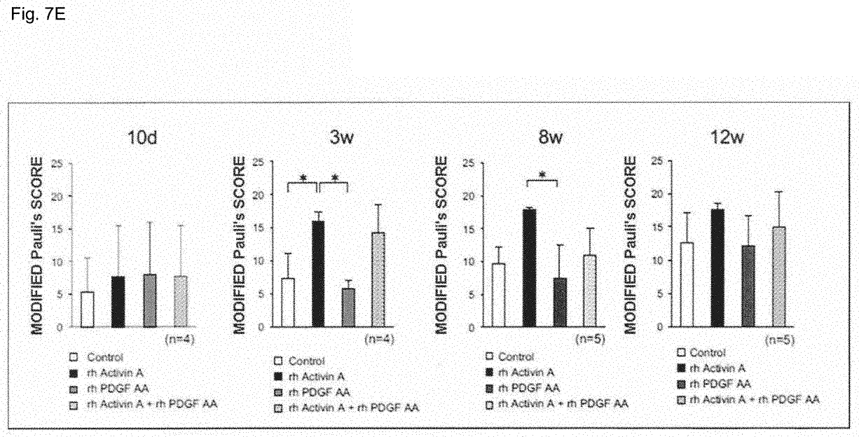

As a result, as shown in FIG. 7A, 10 days after resection (day 3 after Activin A administration>, it was found that uniform granulation, that is, more uniform regenerated meniscus tissue, was induced in the Activin A-administered group. It should also be noted that the vascular invasion in the regenerated region observed in the control and PDGF-administered groups was almost completely suppressed in the Activin A-administered group. In addition, the staining property of safranin O, which stains proteoglycans, a cartilage matrix, was also enhanced compared to the control and PDGF groups.

In addition, as shown in FIG. 7B, 21 days after resection (day 14 after Activin A administration), uniform regeneration of cartilage-like tissue was observed in the Activin A-administered group. Uniform expression of type II collagen, a cartilage matrix, was also observed. It has been reported that vascular invasion has a suppressive effect on the formation of vitreous cartilage. Therefore, it has been suggested that activin functions as an unprecedented inducer of hyaline cartilage.

In addition, as shown in FIG. 7C, 56 days after resection <day 49 after Activin A administration>, the formation of bone marrow-like tissue, which is also observed in normal meniscus of mice, was observed in the center of the uniform cartilage-like tissue in the Activin A-administered group.

Furthermore, as shown in FIG. 7D, 84 days after resection (day 77 after Activin A administration), a regenerated tissue image very close to that of a normal meniscus was observed in the Activin A-administered group. It should be noted that, unlike humans, the meniscus of rodents, including mice, forms a bone marrow cavity in the deep layer of the anterior segment (in humans, the formation of a bone marrow cavity is not observed and the tissue becomes fibrocartilage-like). In the Activin A-administered group, proteoglycan-rich cartilaginous tissue is observed in which the entire surface layer of the regenerated meniscus is stained darkly with safranin O, and the formation of a bone marrow cavity is observed in the deeper layer. This histological image closely resembles the normal histological image of the anterior segment of the meniscus in mice, suggesting that activin is a substance that significantly promotes regeneration after resection in mice and is a molecule that can suppress degeneration for a long time. In addition, no osteoinduction was observed in regenerated tissues administered with activin. Thus, unlike FGF18, which has cartilage regenerative capacity but is concerned about endochondral ossification, activin suppresses ectopic osteoinduction, suggesting that it can regenerate cartilaginous tissue with fewer side effects.

In addition, the histological images of the meniscus at each time point were semi-quantitatively evaluated using a modified Pauli's score. The results showed that the regeneration process was significantly accelerated by administration of Activin A, as shown in FIG. 7E.

### (Example 4) Full Thickness Articular Cartilage Defect Model

Next, in order to verify the regeneration of damaged articular cartilage and the suppression of degenerative degeneration, a model of full thickness articular cartilage defect was prepared to examine the effects of rh Activin A administration. Specifically, a 1.4 mmφ total articular cartilage loss was prepared in the femoral intercondylar region using an airtome, and 180 ng of Activin A (rh Activin A) was injected intra-articularly 1 week later.

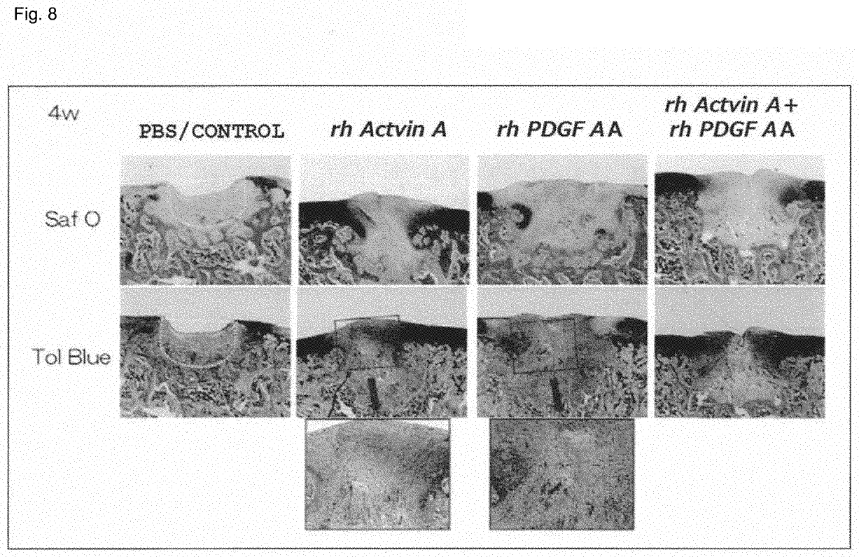

As a result, as shown in FIG. 8, 4 weeks after the defect (week 3 after Activin A administration), both Activin A and PDGF were observed to promote the formation of fibrocartilage-like tissue in the articular cartilage defect site compared to the control (PBS), but no significant promotion effect on chondrogenic differentiation of the formed tissue was observed. However, it should be noted that the degenerative degeneration of articular cartilage around the site of the defect prepared was significantly suppressed in the Activin A-administered group (no decrease in the staining property of safranin O was observed). This result suggests that activin not only has a regeneration-promoting effect on cartilage tissue, but also has a suppressive effect on the degenerative degeneration of cartilage observed in knee osteoarthritis.

### (Example 5) MIA-Induced Arthritis Model

In order to develop activin as a novel therapeutic drug for arthropathy, it is desirable that it not only suppresses cartilage degeneration as described above, but also has a relieving effect on pain, which is the most common complaint of arthropathy.

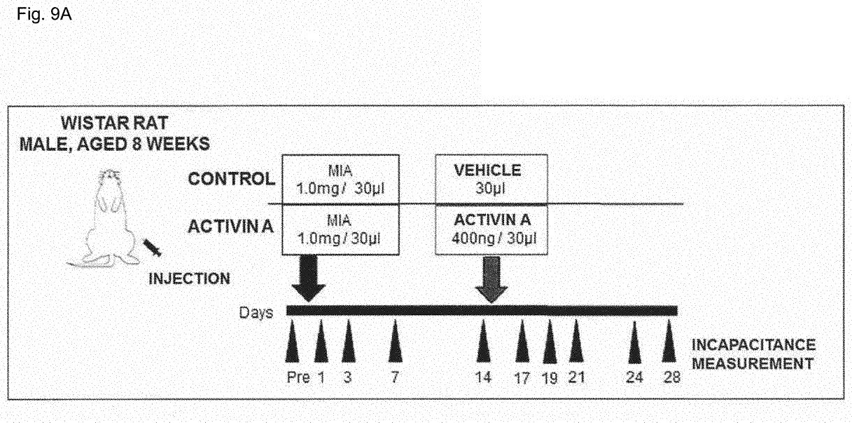

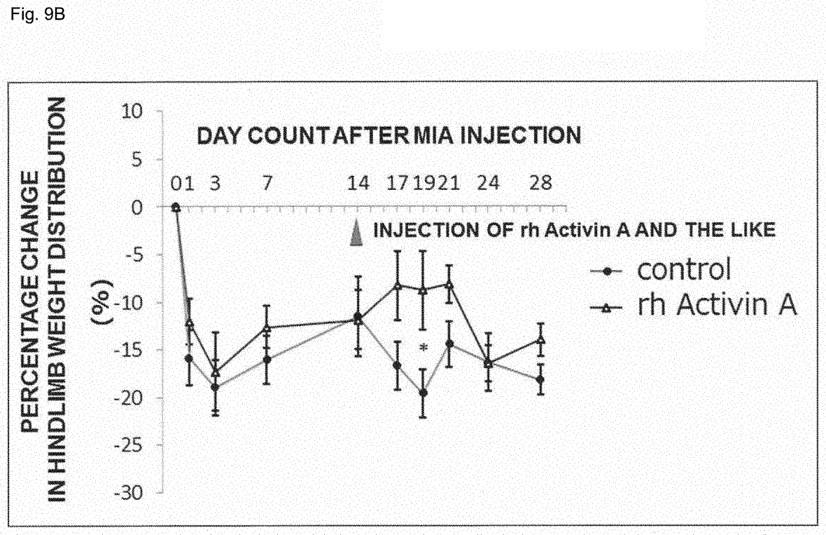

Therefore, an arthritis model by intra-articular injection of mono-iodoacetic acid (MIA) was prepared to investigate the relieving effect of Activin A on knee pain associated with joint inflammation. Specifically, as shown in FIG. 9A, intra-articular injection of Activin A (rh Activin A) was performed at day 14 after MIA administration (inflammation regression period), and the laterality in the load on the hindlimbs of rats was measured over time (Incapacitance test) before and at day 1 after administration.

As a result, as shown in FIG. 9B, intra-articular injection of MIA (left knee) caused a rapid decrease in the load, but when Activin A was administered at day 14, a significant improvement in the load was observed compared to the control group. This indicates that intra-articular administration of Activin A is effective for pain relief.

### (Example 6) Prognosis Prediction by Intra-Articular Activin Concentration

As mentioned above, it is suggested that patients with higher intra-articular concentrations of activin after joint invasion are more likely to have a better prognosis. In view of the above, a correlation analysis was performed between intra-articular Activin A concentration and prognosis using synovial fluid from patients undergoing anterior cruciate ligament reconstruction.

The results showed that patients with higher concentrations of Activin A in synovial fluid subjected to arthrocentesis on postoperative days 3 to 4 had a higher index of patient-based outcome (IKDC) at 2 months postoperatively. Thus, since the concentration of activin in synovial fluid in the early stages after joint invasion increases the index for the recovery of patient-based outcomes in the subsequent stages, it is possible to provide a new diagnostic tool in the field of clinical examinations as a method for predicting the prognosis of joint lesions using the concentration of activin in synovial fluid as an index.

### INDUSTRIAL APPLICABILITY

As described above, the present invention makes it possible to promote the proliferation of MSCs and the regeneration of meniscus or cartilaginous tissue without affecting differentiation multipotency. The invention also makes it possible to treat and prevent meniscus damage or cartilage disorders, including suppression of degenerative degeneration of cartilage and pain. Furthermore, the present invention makes it possible to predict the prognosis of cartilage disorders. Therefore, the present invention is extremely useful in pharmaceutical other fields related to cartilage disorders such as meniscus damage, meniscus defects, articular cartilage defects, degenerative degeneration of articular cartilage, and osteoarthritis.

1. A composition for promoting regeneration of cartilaginous tissues, comprising: activin as an active ingredient.

2. A composition for promoting proliferation of mesenchymal stem cells, comprising: activin as an active ingredient.

3. The composition according to claim 1, which is a composition for treating or preventing a cartilage disorder.

4. The composition according to claim 3, wherein the cartilage disorder is due to damage or defect of a meniscus.

5. The composition according to claim 3, wherein the cartilage disorder is due to defect or degenerative degeneration of articular cartilage.

6. A pharmaceutical composition for treating and/or preventing meniscus damage or cartilage disorder, comprising: activin as an active ingredient.

7. The composition according to claim 6, wherein the meniscus damage is meniscus tear, meniscus degeneration, sparing meniscus degeneration, meniscus defect, meniscus damage associated with accident or sports injury, or meniscus damage associated with aging.

8. The composition according to claim 6, wherein the cartilage disorder is cartilage defect, cartilage damage, cartilage degeneration, cartilage wear, cartilage loss, cartilage degradation, cartilage deformation, defect of articular cartilage, degenerative degeneration of articular cartilage, osteoarthritis, knee osteoarthritis, hip osteoarthritis, shoulder osteoarthritis, traumatic cartilage damage, traumatic cartilage defect, ligamentous damage complication, osteochondritis dissecans, aseptic necrosis, or intervertebral disc damage.

9. The pharmaceutical composition according to claim 6 for administration before, during, and/or after meniscal suture or meniscal repair.

10. The pharmaceutical composition according to claim 6, wherein the activin is Activin A.

11. The pharmaceutical composition according to claim 6, which is used in combination with mesenchymal stem cells.

12. A method for treating or preventing cartilage disorders, comprising the step of: administering activin to a subject's cartilage disorder site.

13. The method according to claim 12, further comprising the step of:
administering mesenchymal stem cells to the cartilage disorder site.

14. A method for predicting prognosis of a cartilage disorder in a joint, comprising the steps of:
measuring a concentration of activin in a joint of a subject; and
predicting that the prognosis of the cartilage disorder in the subject is good if the concentration obtained in the step is higher than a reference value.

15. A composition for promoting proliferation of synovium-derived mesenchymal stem cells in cartilage defect patients or osteoarthritis patients, comprising: activin as an active ingredient.

16. A composition for promoting regeneration of cartilage tissues, comprising: only activin as an active ingredient.

17. A pharmaceutical composition for treating and/or preventing meniscus damage or cartilage disorders, comprising: only activin as an active ingredient.

18. A method for predicting prognosis of a cartilage disorder in a joint, comprising the steps of:
measuring a concentration of activin in a joint during an acute inflammatory phase of a subject; and
predicting that the prognosis of the cartilage disorder in the subject is good if the concentration obtained in the step is higher than a reference value.
